(19) United States
(12) Patent Application Publication
(10) Pub. No.: US 2026/0041623 A1
(43) Pub. Date: Feb. 12, 2026

(54) COSMETIC COMPOSITION COMPRISING ANIONIC AND/OR AMPHOTERIC SURFACTANTS, AT LEAST TWO DIFFERENT CATIONIC POLYSACCHARIDES AND ONE OR MORE SILICONES

(71) Applicant: L'OREAL, Paris (FR)

(72) Inventors: Sophie BOUREL, Saint-Ouen (FR); Adrien BENAZZOUZ, Saint-Ouen (FR); Aldo PIZZINO, Saint-Ouen (FR)

(73) Assignee: L'OREAL, Paris (FR)

(21) Appl. No.: 19/101,643

(22) PCT Filed: Jul. 7, 2023
(86) PCT No.: PCT/EP2023/068913

§ 371 Date: Feb. 6, 2025

(30) Foreign Application Priority Data
Aug. 19, 2022 (FR) FR2208393

(51) Int. Cl. A61K 8/898; A61K 8/41; A61K 8/42; A61Q 5/12

(52) U.S. Cl. CPC A61K 8/898; A61K 8/416; A61K 8/42; A61Q 5/12

(57) ABSTRACT

The present invention relates to a cosmetic composition comprising one or more anionic and/or amphoteric surfactants; at least two cationic surfactants different from one another, and one or more silicones, with a particular weight ratio between the total content of cationic surfactants and the total content of silicone.

The invention also relates to a process for the cosmetic treatment of keratin materials, notably the hair, using said composition.

### RELATED APPLICATIONS

This application is a National Stage Application which claims the benefit of and priority to International Application No. PCT/EP2023/068913 FILED Jul. 7, 2023 which claimed priority to French Application No. 2208393 filed on Aug. 19, 2022, which are hereby incorporated by reference in their entireties.

### DESCRIPTION

The present invention relates to a cosmetic composition comprising at least two cationic surfactants which are different from one another, one or more anionic and/or amphoteric surfactants, and one or more silicones, with a particular weight ratio between the cationic surfactants and the silicones.

The invention also relates to a process for the cosmetic treatment of keratin materials using said composition.

The hair is sensitive to many types of attack and can be embrittled and damaged to various extents by the external environment, in particular by the action of atmospheric agents such as light, atmospheric pollution and bad weather, and also by mechanical or chemical treatments, such as brushing, combing, dyeing, bleaching, permanent-waving and/or relaxing, or even repeated washing.

The properties of damaged hair are then degraded and various manifestations of the damage can be seen, such as difficulties in disentangling and in styling the head of hair, a lack of softness of the hair, hair which can over time become dry, brittle or dull, in particular at fragile places, and more particularly at the ends.

To overcome these drawbacks, it is common practice to make use of hair compositions for conditioning the hair by giving it satisfactory cosmetic properties. These haircare compositions, intended to be applied regularly to the hair, may be, for example, shampoos, conditioners, masks or sera, and may be in the form of gels, hair lotions or care creams that are more or less thick. These compositions are generally based on the use of high contents of silicones and offer benefits of which the extent is rarely sufficient for the most damaged hair and which can be seen only at the time of application and for just a few hours following said application.

Although there are numerous care formulas for hair repair, they do not generally give the hair entirely satisfactory and long-lasting cosmetic properties. Indeed, the benefits provided, such as disentangling, smoothness, sheen, suppleness, or repair of the ends, are not always sufficient for the most sensitive hair or accompanied by a lank feel. Furthermore, these benefits diminish rapidly over time.

In addition, the use of high silicone contents can be viewed unfavorably by consumers.

Therefore, the objective of the present invention is to provide cosmetic compositions for treating keratin materials, notably for conditioning the hair, said composition comprising low silicone contents, in relation to the contents of conditioning agents of cationic surfactant type, said composition being effective in terms of cosmetic properties given to the hair, both at the time of application and for a long-lasting period over time. The composition is also characterized by very marked care and repair benefits while at the same time maintaining added value in terms of lightness and cleanliness.

The aim of the invention is therefore notably to propose a composition capable of providing significant care and lightness properties as soon as it is applied and until the next shampoo wash, using low silicone contents.

A subject of the present invention is a cosmetic composition comprising:
one or more surfactants chosen from anionic surfactants and amphoteric surfactants,
at least two cationic surfactants different from one another, and
one or more silicones,
   the weight ratio between the total content of cationic surfactants and the total content of silicone(s) being greater than or equal to 7.

It is noted that the composition according to the invention rinses off easily, and after application confers a good level of care and also a clean feel of the hair, without making the hair feel lank; these effects are long-lasting for at least 24 hours and can persist after a shampoo wash.

The cosmetic performance results observed, notably in terms of easier disentangling, a greater long-lasting smooth feel, increased suppleness, improved care/nutrition provided, coating quality, repair, in particular of the ends, protection against breaking, and prevention of frizziness, can advantageously be obtained with a low silicone content, notably a content lower than the content normally used.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;

### Cationic Surfactants

The composition comprises at least two cationic surfactants different from one another. It can comprise more than two cationic surfactants, for example at least three cationic surfactants, different from one another.

The two cationic surfactants are different from one another but can belong to the same chemical family, notably to one of the families defined below, and/or can be structurally similar.

The cationic surfactants that can be used according to the present invention are non-silicone surfactants, that is to say that they do not contain any Si—O groups.

They are preferably chosen from primary, secondary or tertiary fatty amines, which are optionally polyoxyalkylenated, or salts thereof, and quaternary ammonium salts, and mixtures thereof.

The cationic surfactants that can be used can advantageously be chosen, alone or as a mixture, from the following compounds, which are quaternary ammonium salts:
the compounds corresponding to the general formula (II) below:

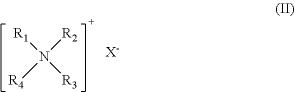

wherein:
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates. the groups R₁ to R₄, which may be identical or different, represent a linear or branched aliphatic group including from 1 to 30 carbon atoms, or an aromatic group such as aryl or alkylaryl, at least one of the groups R₁ to R₄ denoting a linear or branched aliphatic group including from 8 to 30 carbon atoms and preferably from 12 to 24 carbon atoms, the aliphatic groups possibly including heteroatoms notably such as oxygen, nitrogen, sulfur and halogens.

The aliphatic groups are for example chosen from C1-C30 alkyl, C1-C30 alkoxy, (C2-C6)polyoxyalkylene, C1-C30 alkylamide, (C12-C22)alkylamido(C2-C6)alkyl, (C12-C22)alkyl acetate and C1-C30 hydroxyalkyl groups.

Among the quaternary ammonium salts of formula (II), the ones that are preferred are tetraalkylammonium salts, for instance dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, dicetyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts, or stearamidopropyldimethyl(myristyl acetate)ammonium salts such as those sold under the name Ceraphyl® 70 by Van Dyk.

It is preferred in particular to use the chloride, bromide or methosulfate salts of these compounds.
quaternary ammonium salts of imidazoline, such as those of formula (III):

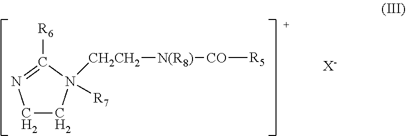

wherein R₅ represents an alkenyl or alkyl group comprising from 8 to 30 carbon atoms, for example derived from tallow fatty acids, R₆ represents a hydrogen atom, a C₁-C₄ alkyl group or an alkenyl or alkyl group comprising from 8 to 30 carbon atoms, R₇ represents a C₁-C₄ alkyl group, R₈ represents a hydrogen atom or a C₁-C₄ alkyl group and X⁻ is an anion chosen from the group of the halides, phosphates, acetates, lactates, alkyl sulfates, alkylsulfonates or alkylarylsulfonates, the alkyl and aryl groups of which preferably comprise, respectively, from 1 to 20 carbon atoms and from 6 to 30 carbon atoms.

Preferably, R₅ and R₆ denote a mixture of alkenyl or alkyl groups including from 12 to 21 carbon atoms, for example derived from tallow fatty acids, R₇ denotes a methyl group and R₈ denotes a hydrogen atom.

Such a product is sold, for example, under the name Rewoquat® W 75 by Rewo,
quaternary diammonium or triammonium salts, in particular of formula (IV):

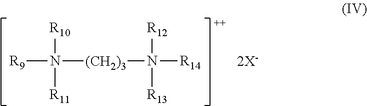

wherein
R₉ denotes an alkyl radical including approximately from 16 to 30 carbon atoms, which is optionally hydroxylated and/or optionally interrupted with one or more oxygen atoms,
R₁₀ is chosen from hydrogen or an alkyl radical including from 1 to 4 carbon atoms or a group (R₉ₐ)(R₁₀ₐ)(R₁₁ₐ)N—(CH₂)₃, with R₉ₐ, R₁₀ₐ, R₁₁ₐ, R₁₁, R₁₂, R₁₃ and R₁₄, which may be identical or different, chosen from hydrogen or an alkyl radical including from 1 to 4 carbon atoms, and
X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, sold by Finetex (Quaternium 89), and Finquat CT, sold by Finetex (Quaternium 75);
quaternary ammonium salts containing at least one ester function, such as those of formula (V) below:

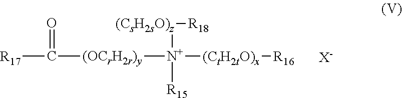

wherein:
R₁₅ is chosen from C₁-C₆ alkyl groups and C₁-C₆ hydroxyalkyl or dihydroxyalkyl groups; R₁₆ is chosen from the group R19-C(O)—; groups R₂₀ which are linear or branched, saturated or unsaturated C₁-C₂₂ hydrocarbon-based groups; a hydrogen atom;
R₁₈ is chosen from the group R21-C(O)—; groups R₂₂ which are linear or branched, saturated or unsaturated C₁-C₆ hydrocarbon-based groups; a hydrogen atom;
R₁₇, R₁₉ and R₂₁, which may be identical or different, are chosen from linear or branched, saturated or unsaturated C₇-C₂₁ hydrocarbon-based groups; r, s and t, which may be identical or different, are integers having values from 2 to 6;
y is an integer ranging from 1 to 10;
x and z, which may be identical or different, are integers having a value from 0 to 10;
X⁻ is a simple or complex, organic or mineral anion;
with the proviso that the sum x+y+z is from 1 to 15, that when x is 0 then R₁₆ denotes R₂₀, and that when z is 0 then R₁₈ denotes R₂₂.

The alkyl groups R₁₅ may be linear or branched, and more particularly linear. Preferably, R₁₅ denotes a methyl, ethyl, hydroxyethyl or dihydroxypropyl group, and more particularly a methyl or ethyl group.

Advantageously, the sum x+y+z is from 1 to 10.

When R16 is a hydrocarbon-based group R20, it may be long and contain from 12 to 22 carbon atoms, or may be short and contain from 1 to 3 carbon atoms.

When R18 is a hydrocarbon-based group R22, it preferably contains 1 to 3 carbon atoms.

Advantageously, R17, R19 and R21, which may be identical or different, are chosen from linear or branched, saturated or unsaturated C11-C21 hydrocarbon-based groups, and more particularly from linear or branched, saturated or unsaturated C11-C21 alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function. The anion X⁻ is even more particularly chloride or methyl sulfate.

In the composition according to the invention, use may be made more particularly of the ammonium salts of formula (V) wherein R15 denotes a methyl or ethyl group, x and y are equal to 1; z is equal to 0 or 1; r, s and t are equal to 2;
R16 is chosen from the group R19-C(O)—, methyl, ethyl or C14-C22 hydrocarbon-based groups, and a hydrogen atom;
R18 is chosen from the group R21-C(O)— and a hydrogen atom;
R17, R19 and R21, which may be identical or different, are chosen from linear or branched, saturated or unsaturated C13-C17 hydrocarbon-based groups, and preferably from linear or branched, saturated or unsaturated C13-C17 alkyl and alkenyl groups.

Advantageously, the hydrocarbon-based groups are linear.

Mention may for example be made of the compounds of formula (V) such as the salts (chloride, bromide or methosulfate notably) of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium. The acyl groups preferably have 14 to 18 carbon atoms and originate more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C10-C30 fatty acids or with mixtures of C10-C30 fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by Henkel, Stepanquat® by Stepan, Noxamium® by CECA or Rewoquat® WE 18 by Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may be made of behenoylhydroxypropyltrimethylammonium chloride sold by KAO under the name Quatarmin BTC 131.

Preferably, the ammonium salts containing at least one ester function contain two ester functions.

Among the quaternary ammonium salts containing at least one ester function that may be used, it is preferred to use dipalmitoylethylhydroxyethylmethyl-ammonium salts, notably the chloride, bromide or methosulfate.

The term “fatty amine” means a compound comprising at least one optionally (poly)oxyalkylenated primary, secondary or tertiary amine function, or salts thereof and comprising at least one C6-C30 and preferably C8-C30 hydrocarbon-based chain.

Preferably, the fatty amines that are useful according to the invention are not (poly)oxyalkylenated.

By way of fatty amines, mention may be made of amidoamines. The amidoamines according to the invention can be chosen from fatty amidoamines, it being possible for the fatty chain to be borne by the amine group or by the amido group.

The term “amidoamine” means a compound comprising at least one amide function and at least one primary, secondary or tertiary amine function.

The term “fatty amidoamine” means an amidoamine comprising, in general, at least one C6-C30 hydrocarbon-based chain. Preferably, the fatty amidoamines that are useful according to the invention are not quaternized.

Preferably, the fatty amidoamines that are useful according to the invention are not (poly)oxyalkylenated.

Preferably, the fatty amidoamines are chosen from (C10-C30)alkylamido(C1-C8)alkyl (di)(C1-C6)alkylamines and salts thereof, better still from (C14-C26)alkylamido(C1-C6)alkyl (di)(C1-C4)alkylamines and salts thereof, preferentially from (C16-C24)alkylamido(C2-C4)alkyl (di)(C1-C2)alkylamines and salts thereof, even more preferentially from (C18-C22)alkylamido(C2-C4)alkyl (di)(C1-C2)alkylamines and salts thereof.

Among the fatty amidoamines of use according to the invention, mention may be made of the amidoamines of formula (VI) below:

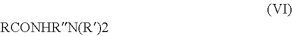

wherein:
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C5-C29 and preferably C7-C23 alkyl radical, or a linear or branched C5-C29 and preferably C7-C23 alkenyl radical;
R″ represents a divalent hydrocarbon-based radical containing less than 6 carbon atoms, preferably from 2 to 4 carbon atoms and better still 3 carbon atoms; and
R′, which may be identical or different, represent a linear or branched, saturated or unsaturated and substituted or unsubstituted monovalent hydrocarbon-based radical containing less than 6 carbon atoms, preferably from 1 to 4 carbon atoms, preferably a methyl radical.

As fatty amidoamines that can be used, mention may be made, alone or as a mixture, of: oleamidopropyl dimethylamine, stearamidopropyl dimethylamine, notably the product sold by Inolex Chemical Company under the name Lexamine S13, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamidopropyl dimethylamine, soyamidopropyl dimethylamine, avocadoamidopropyl dimethylamine, cocamidopropyl dimethylamine, minkamidopropyl dimethylamine, oatamidopropyl dimethylamine, sesamidopropyl dimethylamine, tallamidopropyl dimethylamine, olivamidopropyl dimethylamine, palmitamidopropyl dimethylamine, stearamidoethyl diethylamine, brassicamidopropyl dimethylamine and salts thereof.

Preferably, the fatty amidoamines are chosen, alone or as a mixture, from the following, and also the salts thereof,
brassicamidopropyl dimethylamine of formula R—C(O)—N(H)—(CH2)3-N(CH3)2 wherein R—C(O) is a fatty acid derived from Brassica campestris seed oil (rapeseed oil), with a majority of behenyl (C22) groups;
stearamidopropyl dimethylamine of formula CH3-(CH2)16-C(O)—N(H)—(CH2)3-N(CH3)2;
behenamidopropyl dimethylamine of formula CH3-(CH2)20-C(O)—N(H)—(CH2)3-N(CH3)2
oleamidopropyl dimethylamine.

Preferably, the composition comprises at least one cationic surfactant chosen from those of formula (II) above, those of formula (V) above, those of formula (VI) above, and mixtures thereof; better still from those of formula (II) or (V) above, and mixtures thereof; even better still from those of formula (V) above.

Preferably, the composition comprises at least two cationic surfactants both chosen from those of formula (II) above, those of formula (V) above, those of formula (VI) above, and mixtures thereof; better at least one of the cationic surfactants being chosen from those of formula (V) above and at least one other of the cationic surfactants being chosen from those of formula (II) and/or (VI) above.

Preferentially, the composition comprises one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms.

Even better still, the composition comprises:
(i) one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms; and
(ii) one or more cationic surfactants chosen from:
(iii) tetraalkylammonium salts, notably dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, dicetyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts; preferably the chloride, bromide or methyl sulfate salts of these compounds; and/or
(iib) the amidoamines of formula (VI) RCONHR″N(R′)2 as defined above.

Preferentially, the composition comprises:
(i) one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms; and
(ii) one or more cationic surfactants chosen from tetraalkylammonium salts, notably dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, dicetyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts; preferably the chloride, bromide or methyl sulfate salts of these compounds.

Even better still, the composition comprises:
one or more cationic surfactants chosen from the salts, notably the chlorides, bromides or methosulfates, of dipalmitoylethylhydroxyethylmethylammonium, and mixtures thereof; and
one or more cationic surfactants chosen from the salts, notably the chlorides, bromides or methosulfates, of behenyltrimethylammonium, of distearyldimethylammonium, of cetyltrimethylammonium, of dicetyldimethylammonium and mixtures thereof.

The composition according to the invention preferably comprises the cationic surfactants in a total amount ranging from 0.2% to 10% by weight, better still from 0.5% to 8% by weight, even better still from 1% to 5% by weight, relative to the total weight of the composition.

Notably, when the composition comprises one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof, preferably of diacyloxyethyldimethylammonium and/or of diacyloxyethylhydroxyethylmethylammonium, the acyl groups preferably having 14 to 18 carbon atoms, it advantageously comprises them in a total amount ranging from 0.1% to 5% by weight, better still from 0.2% to 4% by weight, even better still from 0.5 to 2% by weight, relative to the total weight of the composition.

When the composition comprises:
(i) one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms; and
(ii) one or more cationic surfactants chosen from tetraalkylammonium salts, notably dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, cetyltrimethylammonium, dicetyldimethylammonium, or benzyldimethylstearylammonium salts, and also palmitylamidopropyltrimethylammonium salts, stearamidopropyltrimethylammonium salts, stearamidopropyldimethylcetearylammonium salts; preferably the chloride, bromide or methyl sulfate salts of these compounds,
   it advantageously comprises the surfactant(s) (i) in a total amount ranging from 0.1% to 5% by weight, better still from 0.2% to 4% by weight, even better still from 0.5% to 2% by weight, relative to the total weight of the composition and the surfactant(s) (ii) in a total amount ranging from 0.1% to 5% by weight, better still from 0.2% to 4% by weight, even better still from 0.5% to 2% by weight, relative to the total weight of the composition.

Even better still, when the composition comprises:
one or more cationic surfactants chosen from the salts, notably the chlorides, bromides or methosulfates, of dipalmitoylethylhydroxyethylmethylammonium, and mixtures thereof; and
one or more cationic surfactants chosen from the salts, notably the chlorides, bromides or methosulfates, of behenyltrimethylammonium, of distearyldimethylammonium, of cetyltrimethylammonium, of dicetyldimethylammonium and mixtures thereof.
   it advantageously comprises the surfactant(s) chosen from dipalmitoylethylhydroxyethylmethylammonium salts and mixtures thereof, in a total amount ranging from 0.1% to 5% by weight, better still from 0.2% to 4% by weight, even better still from 0.5% to 2% by weight, relative to the total weight of the composition, and
   the surfactant(s) chosen from the behenyltrimethylammonium, distearyldimethylammonium, cetyltrimethylammonium and dicetyldimethylammonium salts and mixtures thereof, in a total amount ranging from 0.1% to 5% by weight, better still from 0.2% to 4% by weight, even better still from 0.5% to 2% by weight, relative to the total weight of the composition.
   Anionic and/or Amphoteric Surfactants

The cosmetic composition according to the invention also comprises one or more surfactants chosen from anionic surfactants and amphoteric surfactants.

The composition can thus comprise one or more anionic surfactants, or one or more amphoteric surfactants, or one or more anionic surfactants and one or more amphoteric surfactants.

Said anionic and said amphoteric surfactants are non-silicone surfactants, that is to say that they do not contain an Si—O group

The term “anionic surfactant” means a surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactants may be sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO—) and may optionally also comprise one or more sulfate and/or sulfonate functions;
the sulfonate anionic surfactants comprise at least one sulfonate function (—SO3H or —SO3-) and may optionally also comprise one or more sulfate functions, but do not comprise any carboxylate functions; and
the sulfate anionic surfactants comprise at least one sulfate function but do not comprise any carboxylate or sulfonate functions.

The carboxylic anionic surfactants that may be used thus include at least one carboxylic or carboxylate function (—COOH or —COO—).

They may be chosen from the following compounds: acylglycinates, acyllactylates, acylsarcosinates, acylglutamates; alkyl ether carboxylic acids, alkyl(C6-30 aryl)ether carboxylic acids, alkyl-D-galactosideuronic acids, alkylamido ether carboxylic acids; and also the salts of these compounds; the alkyl and/or acyl groups of these compounds including from 6 to 30 carbon atoms, notably from 12 to 28, better still from 14 to 24 or even from 16 to 22 carbon atoms; the aryl group preferably denoting a phenyl or benzyl group; these compounds possibly being polyoxyalkylenated, notably polyoxyethylenated, and then preferably including from 1 to 50 ethylene oxide units, better still from 2 to 10 ethylene oxide units.

Use may also be made of the C6-C24 alkyl monoesters of polyglycoside-polycarboxylic acids, such as C6-C24 alkyl polyglycoside-citrates, C6-C24 alkyl polyglycoside-tartrates and C6-C24 alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those including from 2 to 50 alkylene oxide and in particular ethylene oxide groups, such as the compounds sold by Kao under the Akypo names.

The polyoxyalkylenated alkyl(amido)ether carboxylic acids that may be used are preferably chosen from those of formula (1):

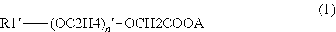

wherein:
R1′ represents a linear or branched C6-C24 alkyl or alkenyl radical, a (C8-C9)alkylphenyl radical, a radical R2′CONH—CH2-CH2- with R2′ denoting a linear or branched C9-C21 alkyl or alkenyl radical; preferably, R1′ is a C8-C20, preferably C8-C18, alkyl radical;
n′ is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (1), in particular mixtures of compounds bearing different groups R1′.

The polyoxyalkylenated alkyl(amido) ether carboxylic acids that are particularly preferred are those of formula (1) wherein:
R1′ denotes a linear or branched C8-C22, in particular C10-C16 or even C12-C14 alkyl radical, or alternatively a (C8-C9)alkylphenyl radical;
A denotes a hydrogen or sodium atom, and
n′ ranges from 2 to 20, preferably from 2 to 10.

Even more preferentially, use is made of the compounds of formula (1) wherein R1′ denotes a C12-C14 alkyl, cocoyl, oleyl, nonylphenyl or octylphenyl radical, A denotes a hydrogen or sodium atom and n′ ranges from 2 to 10.

Among the commercial products that may preferably be used are the products sold by KAO under the names:
Akypo® NP 70 (R1=nonylphenyl, n=7, A=H)
Akypo® NP 40 (R1=nonylphenyl, n=4, A=H)
Akypo® OP 40 (R1=octylphenyl, n=4, A=H)
Akypo® OP 80 (R1=octylphenyl, n=8, A=H)
Akypo® OP 190 (R1=octylphenyl, n=19, A=H)
Akypo® RLM 38 (R1=(C12-C14)alkyl, n=4, A=H)
Akypo® RLM 38 NV (R1=(C12-C14)alkyl, n=4, A=Na)
Akypo® RLM 45 CA (R1=(C12-C14)alkyl, n=4.5, A=H)
Akypo® RLM 45 NV (R1=(C12-C14)alkyl, n=4.5, A=Na)
Akypo® RLM 100 (R1=(C12-C14)alkyl, n=10, A=H)
Akypo® RLM 100 NV (R1=(C12-C14)alkyl, n=10, A=Na)
Akypo® RLM 130 (R1=(C12-C14)alkyl, n=13, A=H)
Akypo® RLM 160 NV (R1=(C12-C14)alkyl, n=16, A=Na),
   or by Sandoz under the names:
Sandopan DTC-Acid (R1=(C13)alkyl, n=6, A=H)
Sandopan DTC (R1=(C13)alkyl, n=6, A=Na)
Sandopan LS 24 (R1=(C12-C14)alkyl, n=12, A=Na)
Sandopan JA 36 (R1=(C13)alkyl, n=18, A=H),
   and more particularly the products sold under the following names:
Akypo® RLM 45 (INCI: Laureth-5 carboxylic acid), Akypo®RLM 100, and Akypo® RLM 38.

Preferentially, the carboxylic anionic surfactants are chosen, alone or as a mixture, from:
acylglutamates, in particular of C6-C24 or even C12-C20, such as stearoylglutamates, and in particular disodium stearoylglutamate;
acylsarcosinates, in particular of C6-C24 or even C12-C20, such as palmitoylsarcosinates, and in particular sodium palmitoylsarcosinate;
acyllactylates, in particular of C12-C28 or even C14-C24, such as behenoyllactylates, and in particular sodium behenoyllactylate;
C6-C24 and in particular C12-C20 acylglycinates;
(C6-C24)alkyl ether carboxylates, and notably (C12-C20)alkyl ether carboxylates; in particular those including from 2 to 50 ethylene oxide groups;
polyoxyalkylenated (C6-C24)alkylamido ether carboxylic acids, in particular those including from 2 to 50 ethylene oxide groups;
   in particular in acid form or in the form of alkali metal or alkaline-earth metal, ammonium or amino alcohol salts.

Polyoxyalkylenated (C6-C24)alkyl ether carboxylic acids and salts thereof are preferably used.

The sulfonate anionic surfactants that may be used include at least one sulfonate function (—SO3H or —SO3-). They may be chosen from the following compounds: alkylsulfonates, alkyl ether sulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds including from 6 to 30 carbon atoms, notably from 12 to 28, better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C6-C24 and in particular C12-C20 olefin sulfonates;
   C6-C24 and in particular C12-C20 alkylsulfosuccinates, in particular laurylsulfosuccinates;
C6-C24 and in particular C12-C20 alkyl ether sulfosuccinates;
(C6-C24)acylisethionates and preferably (C12-C18)acylisethionates; in particular in the form of alkali metal or alkaline-earth metal, ammonium or amino alcohol salts.

The sulfate anionic surfactants that may be used include at least one sulfate function (—OSO3H or —OSO3-).

They may be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds including from 6 to 30 carbon atoms, notably from 12 to 28, better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Preferentially, the sulfate anionic surfactants are chosen, alone or as a mixture, from:
alkyl sulfates, in particular of C6-C24 or even C12-C20, and
alkyl ether sulfates, in particular of C6-C24 or even C12-C20, preferably comprising from 1 to 20 ethylene oxide units;
   in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-propane-1,3-diol salts and tris(hydroxymethyl)aminomethane salts.

Alkali metal or alkaline-earth metal salts and in particular sodium or magnesium salts are preferably used.

Preferentially, the anionic surfactants are chosen, alone or as a mixture, from:
C6-C24 and in particular C12-C20 alkyl sulfates;
C6-C24 and in particular C12-C20 alkyl ether sulfates; preferably comprising from 1 to 20 ethylene oxide units;
C6-C24 and notably C12-C20 alkylsulfosuccinates;
C6-C24 and in particular C12-C20 olefin sulfonates;
C6-C24 and in particular C12-C20 alkyl ether sulfosuccinates;
(C6-C24)acylisethionates and preferably (C12-C18)acylisethionates;
C6-C24 and notably C12-C20 acylsarcosinates;
(C6-C24)alkyl ether carboxylates, preferably (C12-C20)alkyl ether carboxylates; in particular those including from 2 to 50 ethylene oxide groups;
polyoxyalkylenated (C6-C24)alkylamido ether carboxylic acids and salts thereof, in particular those including from 2 to 50 alkylene oxide and in particular ethylene oxide groups;
C6-C24 and in particular C12-C20 acylglutamates;
C6-C24 and in particular C12-C20 acylglycinates;
   in particular in acid form or in the form of alkali metal or alkaline-earth metal, ammonium or amino alcohol salts.

Preferentially, the composition according to the invention comprises one or more anionic surfactants chosen from C6-C24 and notably C12-C20 alkyl sulfates, C6-C24 and notably C12-C20 alkyl ether sulfates preferably comprising from 1 to 20 ethylene oxide units; these surfactants being more particularly in the form of alkali metal or alkaline-earth metal, ammonium or amino alcohol salts; and also mixtures thereof.

The composition according to the invention may also be free (0%) of sulfated surfactants (sulfate free); in this case, it advantageously comprises one or more anionic surfactants chosen from, alone or as a mixture,
C6-C24 and notably C12-C20 alkylsulfosuccinates;
C6-C24 and in particular C12-C20 olefin sulfonates;
C6-C24 and in particular C12-C20 alkyl ether sulfosuccinates;
(C6-C24)acylisethionates and preferably (C12-C18)acylisethionates;
C6-C24 and notably C12-C20 acylsarcosinates;
(C6-C24)alkyl ether carboxylates, preferably (C12-C20)alkyl ether carboxylates; in particular those including from 2 to 50 ethylene oxide groups;
   in particular in acid form or in the form of alkali metal or alkaline-earth metal, ammonium or amino alcohol salts.

When they are present, the anionic surfactant(s) are preferably present in the composition according to the invention in a total amount of less than or equal to 1% by weight, better still (strictly) less than 1% by weight, notably being able to range from 0.001% to 1% by weight, notably from 0.005% to 0.5% by weight, better still from 0.01% to 0.3% by weight, relative to the total weight of the composition.

The amphoteric surfactant(s) that can be used in the context of the invention are nonsilicone.

The term “amphoteric surfactant” means a surfactant comprising, as ionic or ionizable groups, one or more anionic groups and one or more cationic groups.

Mention may in particular be made, alone or as a mixture, of (C8-C20)alkylbetaines, (C8-C20)alkylsulfobetaines, (C8-C20)alkylamido(C3-C8)alkylbetaines and (C8-C20)alkylamido(C6-C8)alkylsulfobetaines.

They may also be chosen from optionally quaternized secondary or tertiary aliphatic amine derivatives, wherein the aliphatic group is a linear or branched chain including from 8 to 22 carbon atoms, said amine derivatives containing at least one anionic group, for instance a carboxylate, sulfonate, sulfate, phosphate or phosphonate group.

Among the optionally quaternized secondary or tertiary aliphatic amine derivatives that can be used, as defined above, mention may also be made of the compounds having the respective structures (A), (B) and (C) below:

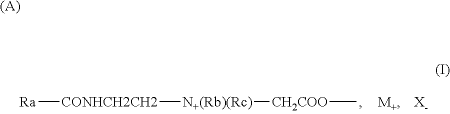

wherein:
Ra represents a C10 to C30 alkyl or alkenyl group derived from an acid RaCOOH preferably present in hydrolyzed coconut kernel oil, or a heptyl, nonyl or undecyl group;
Rb represents a β-hydroxyethyl group; and
Rc represents a carboxymethyl group;
M+ represents a cationic counterion derived from an alkali metal or alkaline-earth metal, such as sodium, an ammonium ion or an ion derived from an organic amine; and
X− represents an organic or mineral anionic counterion, such as that chosen from halides, acetates, phosphates, nitrates, (C1-C4)alkyl sulfates, (C1-C4)alkyl- or (C1-C4)alkylaryl-sulfonates, in particular methyl sulfate and ethyl sulfate; or alternatively M+ and X− are absent;

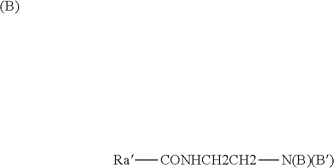

wherein:
B represents the group —CH2CH2OX′;
B′ represents the group —(CH2)zY′, with z=1 or 2;
X′ represents the group —CH2COOH, —CH2-COOZ′, —CH2CH2COOH or CH2CH2-COOZ′, or a hydrogen atom;
Y′ represents the group —COOH, —COOZ′ or —CH2CH(OH)SO3H or the group CH2CH(OH)SO3-Z′;
Z′ represents a cationic counterion derived from an alkali metal or alkaline-earth metal, such as sodium, an ammonium ion or an ion derived from an organic amine;
Ra′ represents a C10 to C30 alkyl or alkenyl group of an acid Ra′—COOH which is preferably present in coconut kernel oil or in hydrolyzed linseed oil, or an alkyl group, in particular a C17 group, and its iso form, or an unsaturated C17 group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by Rhodia under the trade name Miranol® C2M Concentrate.

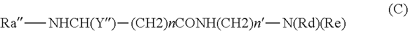

wherein:
Y″ represents the group —COOH, —COOZ″ or —CH2-CH(OH)SO3H or the group CH2CH(OH)SO3-Z″;
Rd and Re, independently of one another, represent a C1 to C4 alkyl or hydroxyalkyl radical;
Z″ represents a cationic counterion derived from an alkali metal or alkaline-earth metal, such as sodium, an ammonium ion or an ion derived from an organic amine;
Ra″ represents a C10 to C30 alkyl or alkenyl group of an acid Ra″-COOH which is preferably present in coconut kernel oil or in hydrolyzed linseed oil;
n and n′ denote, independently of one another, an integer ranging from 1 to 3.

Among the amphoteric surfactants, use is preferably made of (C8-C20)alkylbetaines such as cocoylbetaine, (C8-C20)alkylamido(C3-C8)alkylbetaines such as cocamidopropylbetaine, and mixtures thereof, and the compounds of formula (C) such as the salts, notably the sodium salt of diethylaminopropyl laurylaminosuccinamate (INCI name: sodium diethylaminopropyl cocoaspartamide).

Preferentially, the amphoteric surfactants are chosen from (C8-C20)alkylbetaines such as cocoylbetaine, (C8-C20)alkylamido(C3-C8)alkylbetaines such as cocamidopropylbetaine, and mixtures thereof.

When they are present, the amphoteric surfactant(s) are preferably present in the composition according to the invention in a total amount of less than or equal to 1% by weight, better still (strictly) less than 1% by weight, notably being able to range from 0.001% to 1% by weight, notably from 0.005% to 0.5% by weight, better still from 0.01% to 0.3% by weight, relative to the total weight of the composition.

Preferably, the composition comprises a total content of surfactant(s), chosen from anionic and amphoteric surfactants, ranging from 0.001% to 1% by weight, notably from 0.005% to 0.5% by weight, better still from 0.01% to 0.3% by weight, relative to the total weight of the composition.

### Silicones

The composition according to the invention comprises one or more silicones, which may be chosen in particular from amino silicones, non-amino silicones and mixtures thereof.

The composition according to the invention may thus comprise one or more non-amino silicones, which may be solid or liquid (at 25° C., 1 atm), and volatile or nonvolatile. Preferably, the non-amino silicones are chosen from nonvolatile liquid silicones.

The non-amino silicones that may be used may be soluble or insoluble in the composition according to the invention; they may be in oil, wax, resin or gum form; silicone oils and gums are preferred.

Silicones are particularly described in detail in Walter Noll's “Chemistry and Technology of Silicones” (1968), Academic Press.

The volatile silicones may be chosen from those with a boiling point of between 60° C. and 260° C. (at atmospheric pressure) and in particular from: i) cyclic polydialkylsiloxanes including from 3 to 7 and preferably 4 to 5 silicon atoms, such as
octamethylcyclotetrasiloxane (D4) and decamethylcyclopentasiloxane (D5). Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia;
cyclocopolymers of the dimethylsiloxane/methylalkylsiloxane type having the chemical structure:

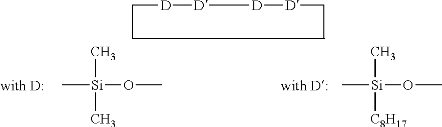

Mention may be made of Volatile Silicone FZ 3109 sold by Union Carbide.
mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;
ii) linear polydialkylsiloxanes containing 2 to 9 silicon atoms, which generally have a viscosity of less than or equal to 5×10-6 m2/s at 25° C., such as decamethyltetrasiloxane.

Other silicones belonging to this category are described in the article published in Cosmetics and Toiletries, Vol. 91, January 76, pages 27-32—Todd & Byers Volatile silicone fluids for cosmetics; mention may be made of the product sold under the name SH 200 by Toray Silicone.

Among the nonvolatile silicones, mention may be made, alone or as a mixture, of polydialkylsiloxanes and notably polydimethylsiloxanes (PDMS or dimethicone), polydiarylsiloxanes, polyalkylarylsiloxanes, silicone gums and resins, and also non-amino organopolysiloxanes (or organomodified polysiloxanes, or alternatively organomodified silicones) which are polysiloxanes including in their structure one or more non-amino organofunctional groups, generally attached via a hydrocarbon-based group, and preferably chosen from aryl groups, alkoxy groups and polyoxyethylene and/or polyoxypropylene groups.

The organomodified silicones may be polydiarylsiloxanes, particularly polydiphenylsiloxanes, and polyalkylarylsiloxanes functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes including:
polyoxyethylene and/or polyoxypropylene groups optionally including C6-C24 alkyl groups, such as dimethicone copolyols, and notably those sold by Dow Corning under the name DC 1248 or the oils Silwet® L 722, L 7500, L 77 and L 711 from Union Carbide; or alternatively (C12)alkylmethicone copolyols, and notably those sold by Dow Corning under the name Q2-5200;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by Goldschmidt;
hydroxylated groups, such as polyorganosiloxanes bearing a hydroxyalkyl function;
acyloxyalkyl groups, such as the polyorganosiloxanes described in U.S. Pat. No. 4,957,732;
anionic groups of the carboxylic acid type, as described, for example, in EP 186 507, or of the alkylcarboxylic type, such as the product X-22-3701 E from Shin-Etsu;
   or else of the 2-hydroxyalkylsulfonate or 2-hydroxyalkylthiosulfate type, such as the products sold by Goldschmidt under the names Abil® S201 and Abil® S255.

The silicones can also be chosen from polydialkylsiloxanes, among which mention may be made mainly of polydimethylsiloxanes having trimethylsilyl end groups (CTFA: dimethicone). Among these polydialkylsiloxanes, mention may be made of the following commercial products:
the Silbione® oils of the 47 and 70 047 series or the Mirasil® oils sold by Rhodia, for instance the oil 70 047 V 500 000;
the oils of the Mirasil® series sold by Rhodia;
the oils of the 200 series from Dow Corning, such as DC200 with a viscosity of 60 000 mm2/s;
the Viscasil® oils from General Electric and certain oils of the SF series (SF 96, SF 18) from General Electric.

Mention may also be made of polydimethylsiloxanes having dimethylsilanol end groups (CTFA: dimethiconol), such as the oils of the 48 series from Rhodia.

In this category of polydialkylsiloxanes, mention may also be made of the products sold under the names Abil Wax® 9800 and 9801 by Goldschmidt, which are poly(C1-C20)dialkylsiloxanes.

Products that may be used more particularly in accordance with the invention are mixtures such as:
mixtures formed from a polydimethylsiloxane with a hydroxy-terminated chain, or dimethiconol (CTFA), and from a cyclic polydimethylsiloxane, also known as cyclomethicone (CTFA), such as the product Q2-1401 sold by Dow Corning.

The polyalkylarylsiloxanes are particularly chosen from linear and/or branched polydimethyl/methylphenylsiloxanes and polydimethyl/diphenylsiloxanes with a viscosity ranging from 1×10-5 to 5×10-2 m2/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

The non-amino silicones that are more particularly preferred according to the invention are polydimethylsiloxanes containing trimethylsilyl end groups (CTFA: dimethicone), which are notably liquid and nonvolatile.

The composition according to the invention may comprise one or more amino silicones.

The term “amino silicone” denotes any silicone including at least one primary, secondary or tertiary amine or a quaternary ammonium group.

The amino silicones that may be used according to the present invention may be volatile or nonvolatile and cyclic, linear or branched, and preferably have a viscosity ranging from 5×10-6 to 2.5 m2/s at 25° C., for example from 1×10-5 to 1 m2/s. Preferably, the amino silicones are chosen from nonvolatile liquid silicones (25° C., 1 atm).

Preferably, the amino silicone(s) are chosen, alone or as mixtures, from the following compounds:
A) the polysiloxanes corresponding to formula (1):

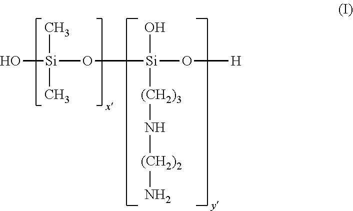

wherein x′ and y′ are integers such that the weight-average molecular mass (Mw) is between 5000 and 500 000 g/mol;
B) the amino silicones corresponding to formula (II):

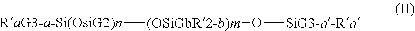

wherein:
G, which is identical or different, denotes a hydrogen atom or a phenyl, OH, C1-C8 alkyl, for example methyl, or C1-C8 alkoxy, for example methoxy, group;
a and a′, which may be identical or different, denote 0 or an integer from 1 to 3, in particular 0, with the proviso that at least one from among a and a′ is equal to zero,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
R′, which may be identical or different, denotes a monovalent radical of formula —CqH2qL wherein q is a number ranging from 2 to 8 and L is an optionally quaternized amino group chosen from the following groups: —NR″-Q-N(R″)2, —N(R″)2, —N+(R″)3 A−, —N+H(R″)2 A−, —N+H2(R″) A−, —NR″-Q-N+(R″)H2 A−, —NR″-Q-N+(R″)2H A- and —NR″-Q-N+(R″)3 A−, wherein R″, which may be identical or different, denotes hydrogen, phenyl, benzyl or a saturated monovalent hydrocarbon-based radical, for example a C1-C20 alkyl radical; Q denotes a linear or branched group of formula CrH2r, r being an integer ranging from 2 to 6, preferably from 2 to 4; and A− represents a cosmetically acceptable anion, notably a halide, such as fluoride, chloride, bromide or iodide.

Preferably, the amino silicones of formula (II) may be chosen from: (i) the “trimethylsilyl amodimethicone” silicones corresponding to formula (III):

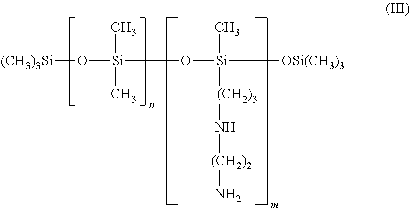

wherein m and n are numbers such that the sum (n+m) varies from 1 to 2000, preferably from 20 to 1000, notably from 50 to 600, better still from 50 to 150; it being possible for n to denote a number from 0 to 1999 and notably from 49 to 149 and it being possible for m to denote a number from 1 to 2000 and notably from 1 to 10;
(ii) the silicones of formula (IV) below:

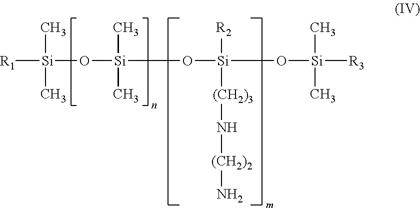

wherein:
m and n are numbers such that the sum (n+m) ranges from 1 to 1000, notably from 50 to 250 and more particularly from 100 to 200; n denoting a number from 0 to 999 and notably from 49 to 249 and more particularly from 125 to 175, and m denoting a number from 1 to 1000, notably from 1 to 10 and more particularly from 1 to 5; and
R1, R2 and R3, which may be identical or different, represent a hydroxyl or C1-C4 alkoxy radical, at least one of the radicals R1 to R3 denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 g/mol and more particularly from 3500 to 200 000 g/mol;
(iii) the silicones of formula (V) below:

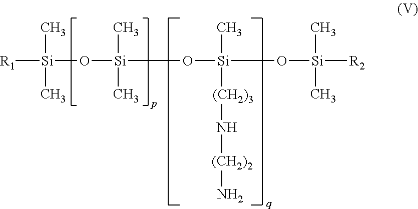

wherein:
p and q are numbers such that the sum (p+q) ranges from 1 to 1000, in particular from 50 to 350 and more particularly from 150 to 250; p denoting a number from 0 to 999, notably from 49 to 349 and more particularly from 159 to 239, and q denoting a number from 1 to 1000, notably from 1 to 10 and more particularly from 1 to 5; and
R1 and R2, which are different, represent a hydroxyl or C1-C4 alkoxy radical, at least one of the radicals R1 or R2 denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) may include in their composition one or more other amino silicones, the structure of which is different from formula (IV) or (V). A product containing amino silicones of structure (IV) is sold by Wacker under the name Belsil® ADM 652. A product containing amino silicones of structure (V) is sold by Wacker under the name Fluid WR 1300®. Another product containing amino silicones of structure (IV) is sold by Wacker under the name Belsil ADM LOG 1®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nm. Preferably, notably as amino silicones of formula (V), use is made of microemulsions of which the mean particle size ranges from 5 nm to 60 nm (limits included) and more particularly from 10 nm to 50 nm (limits included). Thus, use may be made according to the invention of the amino silicone microemulsions of formula (V) sold under the names Finish CT 96 E® or SLM 28020® by Wacker;
(iv) the silicones of formula (VI) below:

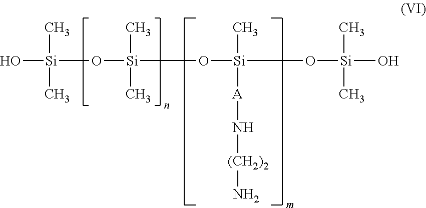

wherein:
m and n are numbers such that the sum (n+m) ranges from 1 to 2000 and notably from 50 to 150, n denoting a number from 0 to 1999 and notably from 49 to 149, and m denoting a number from 1 to 2000 and notably from 1 to 10; and
A denotes a linear or branched alkylene radical having from 4 to 8 carbon atoms and preferably 4 carbon atoms. This radical is preferably linear.

The weight-average molecular mass (Mw) of these amino silicones preferably ranges from 2000 to 1 000 000 g/mol and more particularly from 3500 to 200 000 g/mol.

A silicone corresponding to this formula is, for example, Xiameter MEM 8299 Emulsion from Dow Corning;
(v) the silicones of formula (VII) below:

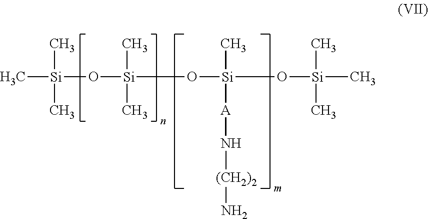

wherein:
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
A denotes a linear or branched alkylene radical having from 4 to 8 carbon atoms and preferably 4 carbon atoms. This radical is preferably branched.

The weight-average molecular mass (Mw) of these amino silicones preferably ranges from 500 to 1 000 000 g/mol and more particularly from 1000 to 200 000 g/mol.

A silicone corresponding to this formula is, for example, DC2-8566 Amino Fluid from Dow Corning;
C) the amino silicones corresponding to formula (VIII):

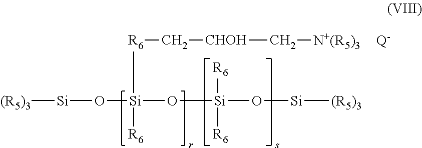

wherein:
R5 represents a monovalent hydrocarbon-based radical having from 1 to 18 carbon atoms, and in particular a C1-C18 alkyl or C2-C18 alkenyl radical, for example methyl;
R6 represents a divalent hydrocarbon-based radical, particularly a C1-C18 alkylene radical or a divalent C1-C18, for example C1-C8, alkyleneoxy radical linked to the Si via an SiC bond;
Q− is an anion such as a halide ion, notably chloride, or an organic acid salt, notably acetate;
r represents a mean statistical value ranging from 2 to 20 and in particular from 2 to 8;
   and
s represents a mean statistical value ranging from 20 to 200 and in particular from 20 to 50.
D) the quaternary ammonium silicones of formula (IX):

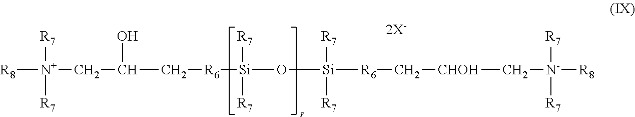

wherein:
R7, which may be identical or different, represent a monovalent hydrocarbon-based radical having from 1 to 18 carbon atoms, and in particular a C1-C18 alkyl radical, a C2-C18 alkenyl radical or a ring comprising 5 or 6 carbon atoms, for example methyl;
R6 represents a divalent hydrocarbon-based radical, particularly a C1-C18 alkylene radical or a divalent C1-C18, for example C1-C8, alkyleneoxy radical linked to the Si via an SiC bond;
R8, which may be identical or different, represent a hydrogen atom, a monovalent hydrocarbon-based radical having from 1 to 18 carbon atoms, and in particular a C1-C18 alkyl radical, a C2-C18 alkenyl radical or a —R6-NHCOR7 radical;
X⁻ is an anion such as a halide ion, notably chloride, or an organic acid salt, notably acetate; and
r represents a mean statistical value ranging from 2 to 200 and in particular from 5 to 100.

These silicones are for example described in patent application EP-A−0 530 974; mention may in particular be made of the silicone having the INCI name: Quaternium 80.

Silicones falling within this category are the silicones sold by Goldschmidt under the names Abil Quat 3270, Abil Quat 3272 and Abil Quat 3474;
E) the amino silicones of formula (X):

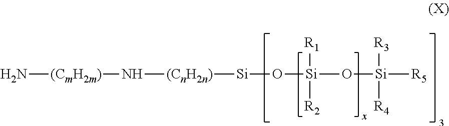

in which:
R1, R2, R3 and R4, which may be identical or different, denote a C1-C4 alkyl radical or a phenyl group,
R5 denotes a C1-C4 alkyl radical or a hydroxyl group,
n is an integer ranging from 1 to 5,
m is an integer ranging from 1 to 5, and
x is chosen such that the amine number ranges from 0.01 to 1 meq/g;
F) the multiblock polyoxyalkylenated amino silicones, of (AB)n type, A being a polysiloxane block and B being a polyoxyalkylenated block including at least one amine group.

Said silicones are preferably formed from repeating units having the following general formulae:

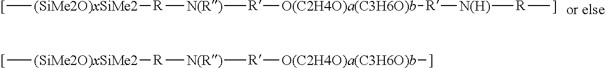

wherein:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C2-C12 hydrocarbon-based radical, optionally including one or more heteroatoms such as oxygen; preferably, R, which may be identical or different, denote an ethylene radical, a linear or branched propylene radical, a linear or branched butylene radical or a CH2CH2CH2OCH2CH(OH)CH2- radical; preferentially, R denote a CH2CH2CH2OCH2CH(OH)CH2- radical; and
R′, which may be identical or different, represent a linear or branched divalent C2-C12 hydrocarbon-based radical, optionally comprising one or more heteroatoms such as oxygen; preferably, R′, which may be identical or different, denote an ethylene radical, a linear or branched propylene radical, a linear or branched butylene radical or a CH2CH2CH2OCH2CH(OH)CH2- radical; preferentially, R′ denote —CH(CH3)-CH2-.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 g/mol and more particularly between 10 000 and 200 000 g/mol.

Mention may particularly be made of the silicones sold under the name Silsoft A−843 or Silsoft A+ by Momentive.
G) the amino silicones of formulae (XI) and (XII):

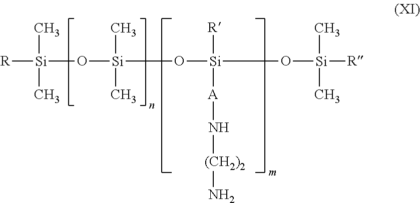

in which:
R, R′ and R″, which may be identical or different, denote a C1-C4 alkyl group or a hydroxyl group,
A denotes a C3 alkylene radical; and
m and n are numbers such that the weight-average molecular mass of the compound is between 5000 and 500 000;

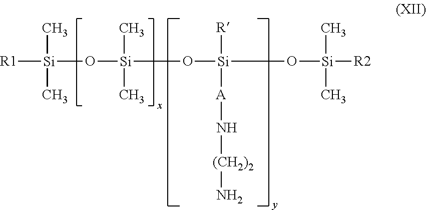

wherein:
x and y are numbers ranging from 1 to 5000; preferably, x ranges from 10 to 2000 and more preferentially from 100 to 1000; preferably, y ranges from 1 to 100;
R1 and R2, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.

Preferably, A comprises from 3 to 6 carbon atoms, more preferentially 4 carbon atoms; preferably, A is branched. Mention may be made in particular of the following divalent groups: —CH2CH2CH2- and —CH2CH(CH3)CH2-.

Preferably, R1 and R2 are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R1 and R2, which may be identical or different, are chosen from hexadecyl (cetyl) and octadecyl (stearyl) groups.

Preferably, in the silicone of formula (XII):
x ranges from 10 to 2000 and in particular from 100 to 1000;
y ranges from 1 to 100;
A comprises from 3 to 6 carbon atoms and notably 4 carbon atoms; preferably, A is branched; more particularly, A is chosen from the following divalent groups: —CH2CH2CH2 and —CH2CH(CH3)CH2-; and
R1 and R2 independently are saturated linear alkyl groups comprising from 6 to 30 carbon atoms, preferably from 8 to 24 carbon atoms and in particular from 12 to 20 carbon atoms; notably chosen from dodecyl, tetradecyl, pentadecyl, hexadecyl, heptadecyl, octadecyl, nonadecyl and eicosyl groups; preferentially, R1 and R2, which may be identical or different, are chosen from hexadecyl (cetyl) and octadecyl (stearyl) groups.

A silicone of formula (XII) that is preferred is bis-cetearyl amodimethicone. Mention may be made in particular of the amino silicone sold under the name Silsoft AX by Momentive.

h) polysiloxanes and notably polydimethylsiloxanes, including primary amine groups at only one chain end or on side chains, such as those of formula (XIV), (XV) or (XVI):

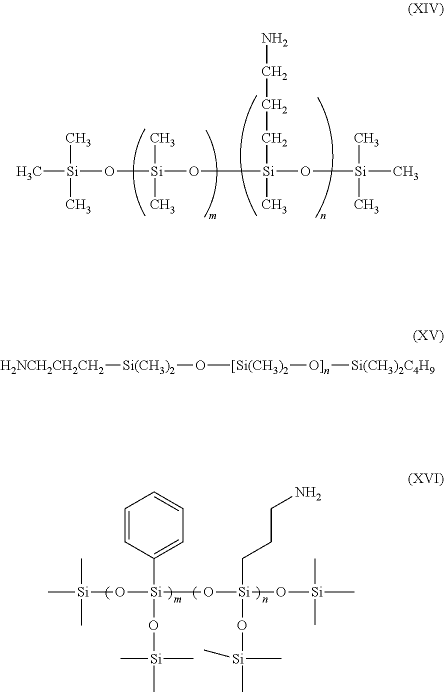

In formula (XIV), the values of n and m are such that the weight-average molecular mass of the amino silicone is between 1000 and 55 000.

As examples of amino silicones of formula (XIV), mention may be made of the products sold under the names AMS-132, AMS-152, AMS-162, AMS-163, AMS-191 and AMS-1203 by Gelest and KF-8015 by Shin-Etsu.

In formula (XV), the value of n is such that the weight-average molecular mass of the amino silicone is between 500 and 3000.

As examples of amino silicones of formula (XV), mention may be made of the products sold under the names MCR-A11 and MCR-A12 by Gelest.

In formula (XVI), the values of n and m are such that the weight-average molecular mass of the amino silicone is between 500 and 50 000.

As examples of amino silicones of formula (XVI), mention may be made of the aminopropyl phenyl trimethicone sold under the name DC 2-2078 Fluid by Dow Corning.

The cosmetic composition according to the invention may also comprise, as silicone, an amino silicone corresponding to formula (XVIII) below:

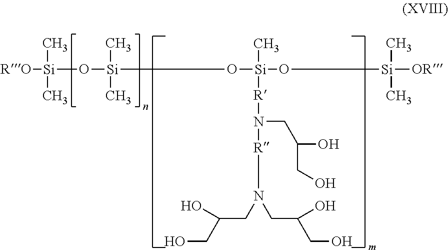

wherein:
n is a number between 1 and 1000, preferably between 10 and 500, better still between 25 and 100, even better still between 50 and 80;
m is a number between 1 and 200, preferably between 1 and 100, better still between 1 and 10 and even better still between 1 and 5;
R′″, which may be identical or different, preferably identical, are linear or branched, saturated or unsaturated alkyl radicals comprising from 8 to 30 carbon atoms, preferably 10 to 24 carbon atoms, notably 12 to 18 carbon atoms; it being possible for said radicals to optionally be substituted with one or more hydroxyl groups OH;
R′ is a linear or branched, divalent alkylene radical having from 1 to 6 carbon atoms, notably from 2 to 5 carbon atoms;
R″ is a linear or branched, divalent alkylene radical having from 1 to 6 carbon atoms, notably from 1 to 5 carbon atoms.

Preferably, the R′″, which may be identical or different, are saturated linear alkyl radicals comprising 8 to 30 carbon atoms, preferably 10 to 24 carbon atoms, notably 12 to 18 carbon atoms; mention may be made in particular of dodecyl, C13, tetradecyl, pentadecyl, hexadecyl, heptadecyl, octadecyl radicals; preferentially, the R′″, which may be identical or different, are chosen from saturated linear alkyl radicals having 12 to 16 carbon atoms, notably which are C13, C14 or C15, alone or as a mixture, and better still represent a mixture of C13, C14 and C15.

Preferably, the R′″ are identical.

Preferably, R′ is a linear or branched, preferably branched, divalent alkylene radical comprising 1 to 6 carbon atoms, notably from 2 to 5 carbon atoms; notably a —CH2-CH2-CH2-, —CH2-CH(CH3)-CH2- or —CH2-CH2-CH(CH3)- radical.

Preferably, R″ is a linear divalent alkylene radical comprising 1 to 6 carbon atoms, notably from 1 to 4 carbon atoms, in particular a —CH2-CH2- radical.

Preferentially, the composition may comprise one or more silicones of formula (XVIII) wherein:
n is a number between 50 and 80;
m is a number between 1 and 5;
R′″, which are identical, are saturated linear alkyl radicals comprising from 12 to 18 carbon atoms;
R′ is a divalent alkylene radical having from 2 to 5 carbon atoms;
R″ is a linear divalent alkylene radical having from 1 to 4 carbon atoms.

Better still, the composition may comprise one or more silicones of formula (XVIII) wherein:
n is a number between 50 and 80;
m is a number between 1 and 5;
R′″, which are identical, are saturated linear alkyl radicals comprising from 13 to 15 carbon atoms;
R′ is a —(CH2)3-, —CH2-CH(CH3)-CH2- or —CH2-CH2-CH(CH3)- radical, and
R″ is a —(CH2)2- radical.

A most particularly preferred silicone of formula (XVIII) is Bis(C13-15 Alkoxy) PG-Amodimethicone (INCI name). Mention may notably be made of the silicone sold under the name Dowsil 8500 Conditioning Agent by Dow.

Preferably, the amino silicone(s) are chosen, alone or as a mixture, from the amino silicones of formulae (I), (III), (VI), (VII), (XI) and/or (XII) defined above.

Preferably, the composition according to the invention comprises one or more amino silicones, better still which are liquid and nonvolatile, notably chosen from the amino silicones of formulae (I), (III), (VI), (VII), (XI) and/or (XII) defined above, even better still from the amino silicones of formulae (1), (111) and/or (XI) as defined above.

Preferably, the composition according to the invention comprises, as silicone, only one or more amino silicones; in other words, according to this preference, the composition does not comprise (comprises 0%) of non-amino silicone.

Advantageously, the composition according to the present invention may comprise the silicone(s) in a total content preferably ranging from 0.01% to 5% by weight, preferentially from 0.05% to 3% by weight, and more preferentially from 0.1% to 2.5% by weight, or even from 0.1% to 1% by weight, even better still from 0.1% to 0.8% by weight, relative to the total weight of the composition.

Advantageously, the composition according to the present invention may comprise the amino silicone(s) in a total content preferably ranging from 0.01% to 5% by weight, preferentially from 0.05% to 3% by weight, and even better still from 0.1% to 2.5% by weight, or even from 0.1% to 1% by weight, better still from 0.1% to 0.8% by weight, relative to the total weight of the composition.

According to the invention, the weight ratio between the total content of cationic surfactants and the total content of silicone(s) present in the composition according to the invention is greater than or equal to 7. Advantageously, this weight ratio may range from 7 to 20, notably from 8 to 15 and even better still from 9 to 13.

Preferably, the weight ratio between the total content of cationic surfactants and the total content of amino silicone(s) present in the composition according to the invention is greater than or equal to 7, better still ranges from 7 to 20, even better still ranges from 8 to 15 and even better still ranges from 9 to 13.

### Solid Fatty Substances

The composition according to the invention may optionally comprise one or more nonsilicone solid fatty substances, which can be chosen advantageously from solid fatty alcohols, solid esters of fatty acids and/or of fatty alcohols, waxes, butters, ceramides, and mixtures thereof.

The term “fatty substance” means an organic compound that is insoluble in water at 25° C., 1 atm, i.e. with a solubility of less than 5% by weight, preferably less than 1% by weight and even more preferentially less than 0.1% by weight. The fatty substances are generally soluble in organic solvents under the same temperature and pressure conditions, for instance chloroform, dichloromethane, carbon tetrachloride, ethanol, benzene, toluene, tetrahydrofuran (THF) or liquid petroleum jelly.

The term “solid fatty substance” means a fatty substance having a melting point of greater than 25° C., preferably greater than or equal to 28° C., better still greater than or equal to 30° C., at atmospheric pressure (1 atm).

Advantageously, the solid fatty substances that may be used in the present invention are neither (poly)oxyalkylenated nor (poly)glycerolated.

“Fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated. The solid fatty alcohols may be saturated or unsaturated, and linear or branched, and include from 8 to 40 carbon atoms, preferably from 10 to 30 carbon atoms, better still from 12 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 12 to 30, or even from 12 to 24 atoms and even better still from 14 to 22 carbon atoms.

The solid fatty alcohols that may be used are preferably chosen from saturated, and linear or branched, preferably linear and saturated, (mono)alcohols including from 8 to 40 carbon atoms, better still from 10 to 30, or even from 12 to 24 atoms and better still from 14 to 22 carbon atoms.

The solid fatty alcohols that may be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol or mixtures thereof such as cetylstearyl alcohol, better still the solid fatty alcohol is cetylstearyl alcohol.

The solid esters of a fatty acid and/or of a fatty alcohol that may be used are preferably chosen from solid esters derived from a C9-C26 carboxylic fatty acid and/or from a C9-C26 fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C4-C22 dicarboxylic or tricarboxylic acids and of C1-C22 alcohols and esters of mono-, di- or tricarboxylic acids and of C2-C26 di-, tri-, tetra- or pentahydroxy alcohols may also be used.

Mention may particularly be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C9-C26 alkyl palmitates, notably myristyl palmitate, cetyl palmitate and stearyl palmitate; C9-C26 alkyl myristates, such as cetyl myristate, stearyl myristate and myristyl myristate; C9-C26 alkyl stearates, notably myristyl stearate, cetyl stearate and stearyl stearate; and mixtures thereof.

Particularly preferably, the solid esters of a fatty acid and/or of a fatty alcohol are chosen from myristyl stearate, myristyl palmitate, and mixtures thereof.

For the purposes of the present invention, a wax is a lipophilic compound, which is solid at 25° C. and atmospheric pressure, with a reversible solid/liquid change of state, having a melting point of greater than approximately 40° C. and which may be up to 200° C., and having anisotropic crystal organization in the solid state. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to ambient temperature, recrystallization of the wax, which is microscopically and macroscopically detectable (opalescence), is obtained.

In particular, the waxes that are suitable for use in the invention may be chosen from waxes of animal, plant or mineral origin, nonsilicone synthetic waxes, and mixtures thereof.

Mention may notably be made of hydrocarbon-based waxes, for instance beeswax or modified beeswaxes (cera bellina), lanolin wax and lanolin derivatives, spermaceti; cork fiber or sugarcane waxes, olive tree wax, rice bran wax, carnauba wax, candelilla wax, ouricury wax, esparto grass wax, berry wax, shellac wax, Japan wax and sumac wax, absolute waxes of flowers; montan wax, orange wax, lemon wax, microcrystalline waxes, paraffins, petroleum jelly, lignite and ozokerite; polyethylene waxes, the waxes obtained by Fischer-Tropsch synthesis and waxy copolymers, and also esters thereof.

Mention may also be made of C2 to C60 microcrystalline waxes, such as Microwax HW. Mention may also be made of the MW 500 polyethylene wax sold under the reference Permalen 50-L Polyethylene. Mention may also be made of the waxes obtained by catalytic hydrogenation of animal or plant oils containing linear or branched C8 to C32 fatty chains. Among these waxes, mention may notably be made of isomerized jojoba oil such as trans-isomerized partially hydrogenated jojoba oil, notably the product manufactured or sold by Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, notably the product sold under the name Hest 2T-4S® by Heterene. The waxes obtained by hydrogenation of castor oil esterified with cetyl alcohol, such as those sold under the names Phytowax Castor 16L64® and 22L73® by Sophim, may also be used.

A wax that may be also used is a C20 to C40 alkyl (hydroxystearyloxy)stearate (the alkyl group comprising from 20 to 40 carbon atoms), alone or as a mixture. Such a wax is particularly sold under the names Kester Wax K 82 P®, Hydroxypolyester K 82 P® and Kester Wax K 80 P® by Koster Keunen.

It is also possible to use microwaxes in the compositions of the invention; mention may notably be made of carnauba microwaxes, such as the product sold under the name MicroCare 350® by Micro Powders, synthetic-wax microwaxes, such as the product sold under the name MicroEase 114S® by Micro Powders, microwaxes constituted of a mixture of carnauba wax and polyethylene wax, such as the products sold under the names Micro Care 300® and 310® by Micro Powders, microwaxes constituted of a mixture of carnauba wax and of synthetic wax, such as the product sold under the name Micro Care 325® by Micro Powders, polyethylene microwaxes, such as the products sold under the names Micropoly 200®, 220®, 220L® and 250S® by Micro Powders, and polytetrafluoroethylene microwaxes, such as the products sold under the names Microslip 519® and 519 L® by Micro Powders.

The waxes are preferably chosen from mineral waxes, for instance paraffin, petroleum jelly, lignite or ozokerite wax; plant waxes, for instance cocoa butter, shea butter or cork fiber or sugar cane waxes, olive tree wax, rice bran wax, hydrogenated jojoba wax, ouricury wax, carnauba wax, candelilla wax, esparto grass wax, or absolute waxes of flowers, such as the essential wax of blackcurrant blossom sold by Bertin (France); waxes of animal origin, for instance beeswaxes or modified beeswaxes (cera bellina), spermaceti, lanolin wax and lanolin derivatives; microcrystalline waxes; and mixtures thereof.

Butters may also be used as solid fatty substance.

For the purposes of the present invention, the term “butter” (also referred to as “pasty fatty substance”) means a lipophilic fatty compound with a reversible solid/liquid change of state and including, at a temperature of 25° C. and at atmospheric pressure (760 mmHg), a liquid fraction and a solid fraction. Preferably, the butter(s) according to the invention have a melting start temperature above 25° C. and a melting end temperature below 60° C.

Preferably, the particular butter(s) are of plant origin, such as those described in Ullmann's Encyclopedia of Industrial Chemistry (“Fats and Fatty Oils”, A. Thomas, published online: Jun. 15, 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

An example of a preferred butter is shea butter. In a known manner, shea butter is extracted from the fruit (also called “kernels” or “nuts”) of the Butyrospermum parkii tree. Each fruit contains between 45% and 55% fatty substance, which is extracted and generally refined. Shea butter contains fatty acid triglycerides. The fatty acid distribution of shea butter is variable, notably depending on the geographical origin of the nuts, the total percentage of stearic and oleic acids being very greatly predominant and generally greater than 80% by weight relative to the total weight of fatty acids.

The ceramides, or ceramide analogs such as glycoceramides, which may be used in the compositions according to the invention, are known; mention may be made in particular of ceramides of classes I, II, III and V according to the Dawning classification.

The ceramides or analogs thereof that may be used preferably correspond to the following formula:

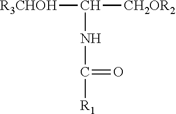

wherein:
R1 denotes a linear or branched, saturated or unsaturated alkyl group, derived from C14-C30 fatty acids, it being possible for this group to be substituted with a hydroxyl group in the alpha position, or a hydroxyl group in the omega position esterified with a saturated or unsaturated C16-C30 fatty acid;
R2 denotes a hydrogen atom, a (glycosyl)n group, a (galactosyl)m group or a sulfogalactosyl group, wherein n is an integer ranging from 1 to 4 and m is an integer ranging from 1 to 8;
R3 denotes a C15-C26 hydrocarbon-based group, which is saturated or unsaturated in the alpha position, it being possible for this group to be substituted with one or more C1-C14 alkyl groups;
it being understood that, in the case of natural ceramides or glycoceramides, R3 may also denote a C15-C26 alpha-hydroxyalkyl group, the hydroxyl group optionally being esterified with a C16-C30 alpha-hydroxy acid.

Preferentially, use is made of ceramides for which R1 denotes a saturated or unsaturated alkyl group derived from C14-C30 fatty acids; R2 denotes a galactosyl or sulfogalactosyl group; and R3 denotes a —CH═CH—(CH2)12-CH3 group.

The ceramides that are more particularly preferred are the compounds for which R1 denotes a saturated or unsaturated alkyl derived from C16-C22 fatty acids; R2 denotes a hydrogen atom and R3 denotes a saturated or unsaturated linear C15 group.

Use may also be made of the compounds for which R1 denotes a saturated or unsaturated alkyl radical derived from C12-C22 fatty acids; R2 denotes a galactosyl or sulfogalactosyl radical; and R3 denotes a saturated or unsaturated C12-C22 hydrocarbon-based radical and preferably a —CH═CH—(CH2)12-CH3 group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

The composition according to the invention preferably comprises one or more nonsilicone solid fatty substances chosen from solid fatty alcohols, solid esters of fatty acids and/or of fatty alcohols, and mixtures thereof.

Advantageously, the composition according to the invention comprises one or more solid fatty alcohols and one or more solid esters of fatty acids and/or of fatty alcohols.

Even better still, the composition according to the invention comprises one or more nonsilicone solid fatty substances chosen from linear or branched, saturated, preferably linear and saturated, (mono)alcohols comprising from 8 to 40 carbon atoms, better still from 10 to 30, or even from 12 to 24 atoms, even better still from 14 to 22 carbon atoms; C9-C26 alkyl palmitates, C9-C26 alkyl myristates, C9-C26 alkyl stearates; and mixtures thereof.

Preferably, the composition according to the invention comprises the nonsilicone fatty substance(s) in a total content ranging from 1% to 15% by weight, preferably from 2% to 10% by weight and preferentially from 3% to 8% by weight, relative to the total weight of the composition.

Preferably, the composition according to the invention comprises the nonsilicone solid fatty substance(s) chosen from solid fatty alcohols, solid esters of fatty acids and/or of fatty alcohols, and mixtures thereof, in a total content ranging from 1% to 15% by weight, preferably from 2% to 10% by weight and preferentially from 3% to 8% by weight, relative to the total weight of the composition.

### Other Ingredients

The composition according to the invention advantageously comprises water, which may be present in a proportion of from 65% to 98% by weight, better still from 70% to 97% by weight and preferably from 75% to 95% by weight, relative to the total weight of the composition.

The composition according to the invention may also comprise, furthermore, one or more water-miscible organic solvents. Preferably, the organic solvent(s) are chosen from non-aromatic C1-C6 alcohols such as ethyl alcohol or isopropyl alcohol, aromatic alcohols such as benzyl alcohol and phenylethyl alcohol, or polyols such as C2-C6, better still C2-C4, diols or triols such as propylene glycol or glycerol. When they are present in the composition according to the invention, the organic solvent(s) generally represent from 0.1% to 15% by weight and preferably from 0.5% to 10% by weight and better still from 1% to 5% by weight, relative to the total weight of the composition.

The cosmetic composition according to the present invention may also optionally comprise one or more additives, different from the compounds of the invention and among which mention may be made of nonionic surfactants, cationic, anionic, nonionic or amphoteric polymers, or mixtures thereof, nonsilicone liquid fatty substances, antidandruff agents, antiseborrhoeic agents, vitamins and provitamins including panthenol, sunscreens, sequestrants, plasticizers, solubilizers, acidifying agents, mineral or organic thickeners, in particular polymeric thickeners, opacifiers, pearlescent agents, antioxidants, hydroxy acids, fragrances, preserving agents and fillers.

Of course, those skilled in the art will take care to choose this or these optional additional compounds such that the advantageous properties intrinsically attached to the composition according to the invention are not, or not substantially, adversely affected by the envisaged addition(s). The above additives can generally be present in an amount, for each of them, of between 0% and 20% by weight, with respect to the total weight of the composition.

A subject of the invention is also a process for the cosmetic treatment of keratin materials, notably human keratin fibers such as the hair, comprising the application to said keratin materials of a composition as defined above.

The composition according to the invention may be applied to dry or wet keratin materials that have optionally been washed with shampoo.

Preferably, the composition according to the invention is applied to wet keratin fibers.

After an optional leave-in time which can range from 1 to 15 minutes, preferably from 2 to 10 minutes, the keratin fibers can then be rinsed, for example water, before being dried or left to dry.

The process for the cosmetic treatment of keratin materials is preferably a hair conditioning process.

The composition according to the invention is preferably in the form of a rinse-out conditioner or of a rinse-out hair mask, but can also be in the form of a leave-in hair product or hair serum.

The examples that follow serve to illustrate the invention without, however, being limiting in nature.

In the examples that follow, all the amounts are given, unless otherwise indicated, as mass percentages of active material (AM) relative to the total weight of the composition.

### EXAMPLE 1

Compositions A to D according to the invention were prepared from the following ingredients (% AM):

TABLE 1

| | A | B | C | D |
|---|---|---|---|---|
| Amodimethicone (DOWSIL | 0.2 | 0.2 | 0.2 | 0.2 |
| AP-8568 AMINO FLUID from | | | | |
| Dow Corning) | | | | |
| Behentrimonium chloride | 0.8 | 1.0 | 1.0 | — |
| Stearamidopropyldimethylamine | — | — | — | 0.95 |
| Dipalmitoylethylhy- | 1.0 | 1.0 | 1.0 | 1.0 |
| droxyethylmonium methosulfate | | | | |
| Cocobetaine | 0.02 | 0.02 | — | 0.02 |
| Sodium laureth sulfate | — | — | 0.015 | — |
| Cetearyl alcohol | 4.7 | 4.6 | 4.6 | 4.6 |
| Cetyl esters | 0.4 | 0.4 | 0.4 | 0.4 |
| Glycerol | — | 2 | — | — |
| pH Agent (pH = 4.1 ± 0.5) | qs | qs | qs | qs |
| Preservatives | qs | qs | qs | qs |
| Water | qs 100 | qs 100 | qs 100 | qs 100 |
| Cationic surfactants/silicone ratio | 9 | 10 | 10 | 9.5 |

Compositions which can be used as a rinse-out hair care product (conditioner) are obtained.

These compositions give the hair good cosmetic properties (conditioning properties), notably in terms of smooth feel, disentangling and sheen; they also impart repair benefits, while at the same time leaving the hair light.

Composition A according to the invention was tested with respect to a commercial conditioner for damaged hair.

A panel of testers, regular users of conditioning hair products for dry, damaged or dyed hair, tested either composition A according to the invention, or the commercial conditioner for dry hair, for 2 weeks.

Composition A (or the commercial conditioner) was used in the normal way (application, leave-in time, rinse-out, drying), after the use of a commercial shampoo.

After 2 weeks of use, the testers indicated that they were in agreement/absolutely in agreement with the proposed performance criterion.

The following results were obtained (% of testers in agreement/absolutely in agreement with the performance criteria):

TABLE 2

| Performance criterion | A (invention) | A′ (commercial) |
|---|---|---|
| Smooth feel to the hair | 80% | 64% |
| Hair shiny | 91% | 85% |
| Hair light until next shampoo wash | 94% | 83% |
| Ease of disentangling | 94% | 89% |
| Protects against breaking | 88% | 76% |
| Makes the hair stronger, more robust | 87% | 79% |

It is noted that, for each of the criteria, the difference is significantly in favor of the composition according to the invention. It thus makes it possible to provide care benefits (disentangling, smoothing, sheen) and repair benefits (protection against breaking, strengthening of the hair) which are significantly greater than a commercially available composition specially intended for damaged hair. It also provides long-lasting lightness added value.

### EXAMPLE 2

Composition E according to the invention and comparative composition E′ were prepared from the following ingredients (% AM):

TABLE 3

| | E | E′ |
|---|---|---|
| Amodimethicone (DOWSIL AP-8568 AMINO FLUID | 0.2 | 0.2 |
| from Dow Corning) | | |
| Behentrimonium chloride | 0.9 | 0.9 |
| Dipalmitoylethylhydroxyethylmonium methosulfate | 1.0 | 1.0 |
| Sodium laureth sulfate | 0.04 | — |
| Cetearyl alcohol | 4.6 | 4.6 |
| Cetyl esters | 0.4 | 0.4 |
| pH Agent (pH = 4.1 ± 0.5) | qs | qs |
| Preservatives | qs | qs |
| Water | qs 100 | qs 100 |

Compositions which can be used as a rinse-out hair care product (conditioner) are obtained.

### Evaluation of the Uniformity of the Deposit

The compositions are tested on locks of medium-sensitized Caucasian hair (SA20) of 1 g and 27 cm in length.

Firstly, the locks are cleaned using a standard silicone-free shampoo (DOP Camomile), in a proportion of 0.4 g of shampoo per g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet locks in a proportion of 0.4 g/g of hair, before a leave-in time of 5 minutes followed by rinsing with water for 10 seconds (flow rate: 300 l/h, temperature: 35° C.).

The uniformity of the silicone deposit is evaluated on wet hair by measuring the frictional forces on the lock treated along the length to the end of the hair, during the movement thereof at constant speed, between two rollers, while squeezing it at constant compression.

The difference in frictional forces between the body of the lock and the end of the lock is determined in order to establish the quality of the deposit. The smaller the difference, the more uniform the deposit on the whole of the hair, reflecting a more qualitative sensation of smooth touch.

The following results are obtained (average of the measurements, carried out on 6 locks):

TABLE 4

| | E (invention) | E′ (comparative) |
|---|---|---|
| Difference (Fends − Flength) | 80 | 140 |

The difference in frictional forces measured on wet hair, between the body of the lock and the end, is significantly smaller for the locks treated with the composition according to the invention.

The surface deposit provided by the composition according to the invention therefore appears to be more uniform and confers a better smooth feel quality on the whole of the hair.

### Sensory Evaluation

The tests are carried out on locks of medium-sensitized Caucasian hair (SA20) of 2.7 g and 27 cm in length. The locks are pre-cleaned using a standard shampoo (DOP Camomile) in a proportion of 0.4 g/g of hair.

Each block is then treated with a conventional repair shampoo (ELSEVE Total Repair 5, L'Oréal Paris) in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). The hair is wrung dry and then evaluated while wet. Finally, the locks are dried with a hairdryer and then evaluated (dry).

The disentangling is evaluated using a comb, whereas the smooth nature and the coating are scored on feel. The scores cover a scale of 0 (lack of performance) to 5 (maximum performance).

Evaluation of the smooth feel: the evaluator takes a lock of hair at the root and slides it through the fingers over the entire length of the lock as far as the ends. The more the hair is uniform, homogeneous from the root to the end, the fewer bumps it exhibits, the less it catches on the fingers, the better the smooth feel.

Evaluation of the coating: the evaluator takes the lock of hair in the hand, starts from the root and slides as far as the ends. The more a deposit is felt on the fiber, the better the coating.

Evaluation of the disentangling: the disentangling is evaluated using a comb that the evaluator slides from the root to the end. The more easily the comb slides, the better the disentangling.

The following results are obtained:

TABLE 5

| | Evaluated criterion | E (invention) | E′ (comparative) |
|---|---|---|---|
| Wet hair | Ease of disentangling | 4.5 | 3.5 |
| | Smooth feel | 4 | 3 |
| | Coating | 4 | 3 |
| Dried hair | Smooth feel | 4 | 3 |
| | Coating | 4 | 3 |

It is noted that the composition according to the invention comprising the anionic surfactant is significantly superior over the whole of the criteria tested, compared to the composition without anionic surfactant. This superiority manifests itself on wet hair through a greater ease of disentangling, hair that feels smoother and a coating that is more present signalling a better transformation of the fiber. On dried hair, the hair appears more treated (greater care coating) and softer (smoother feel).

### EXAMPLE 3

Composition F according to the invention and comparative composition F′ were prepared from the following ingredients (% AM):

TABLE 6

| | F | F′ |
|---|---|---|
| Amodimethicone (DOWSIL AP-8568 AMINO FLUID | 0.24 | 0.71 |
| from Dow Corning) | | |
| Behentrimonium chloride | 1.2 | 1.2 |
| Dipalmitoylethylhydroxyethylmonium methosulfate | 1.3 | 1.3 |
| Cocobetaine | 0.03 | 0.03 |
| Cetearyl alcohol | 5.2 | 5.2 |
| Cetyl esters | 0.4 | 0.4 |
| pH Agent (pH = 4.1 ± 0.5) | qs | qs |
| Preservatives | qs | qs |
| Water | qs 100 | qs 100 |
| Cationic surfactants/silicone ratio | 10.4 | 3.5 |

Compositions which can be used as a rinse-out hair care product (conditioner) are obtained.

### Deposit on Hair

The compositions are tested on locks of natural Caucasian hair of 2.7 g and 27 cm in length.

Firstly, the locks are cleaned using a standard silicone-free shampoo (DOP Camomile), in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). Finally, the locks are dried for 30 minutes in an oven at 60° C.

The cycle of application of standard shampoo+composition to be tested+drying in an oven is repeated a further 4 times.

The deposit of the silicone on the hair is quantified by X-ray fluorescence spectrometry via the measurement of the mass concentration of silicon element on the hair fiber. The amount of silicon naturally present in the untreated hair is subtracted from the measurement obtained on the treated hair (silicon deposit=content measured on treated hair−content measured on untreated hair).

The following results are obtained (silicon deposit measured by XRF on dry hair treated by multiple applications (×5) with compositions F or F′. The values are expressed in μg of silicon element per g of hair):

TABLE 7

| Silicon Deposit | F (invention) | F′ (comparative) |
|---|---|---|
| Mean value | 251 | 33 |
| Standard deviation | 50 | 24 |

It is noted that the composition according to the invention results in a significantly greater silicone deposit on the hair than the comparative composition, even though it comprises a lower silicone concentration.

### Sensory Evaluation

The tests are carried out on locks of medium-sensitized Caucasian hair (SA20) of 2.7 g and 27 cm in length. The locks are pre-cleaned using a standard shampoo (DOP Camomile) in a proportion of 0.4 g/g of hair.

Each block is then treated with a conventional repair shampoo (ELSEVE Total Repair 5, L'Oreal Paris) in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). The hair is wrung dry and then evaluated while wet. Finally, the locks are dried with a hairdryer and then evaluated (dry).

The disentangling is evaluated using a comb (by 1 evaluator), whereas the smooth nature is scored on feel (by 4 evaluators). The scores cover a scale of 0 (lack of performance) to 5 (maximum performance).

The following results are obtained:

TABLE 8

| | Evaluated criterion | F (invention) | F′ (comparative) |
|---|---|---|---|
| Wet hair | Ease of disentangling | 4.5 | 4.0 |
| | Smooth feel | 3.8 ± 0.2 | 2.5 ± 0.6 |
| Dried hair | Smooth feel | 4.2 ± 0.3 | 3.3 ± 0.4 |

It is noted that the composition according to the invention facilitates the disentangling and confers performance superiority in terms of smoothing of the hair in both wet and dry conditions, which are significantly superior to those of the comparative composition, even though it comprises less silicone.

### Instrumental Evaluation of Smoothing Performance

The compositions are tested on locks of medium-sensitized Caucasian hair (SA20) of 1 g and 27 cm in length.

Firstly, the locks are cleaned using a standard silicone-free shampoo (DOP Camomile), in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 5 minutes followed by rinsing with water for 15 seconds (flow rate: 300 l/h, temperature: 35° C.).

The frictional forces on dry hair, of the lock treated along the length and as far as the end of the hair, during the movement thereof at constant speed, between two rollers, while squeezing it at constant compression are measured. Low frictional forces reflect a smoother surface condition of the hair.

The following results are obtained (average of the measurements, carried out on 6 locks):

TABLE 9

| Frictional forces (mJ) | F (invention) | F′ (comparative) |
|---|---|---|
| Mean of frictional forces | 143.4 | 164.5 |
| Standard deviation | 5.6 | 6.6 |

It is noted that the composition according to the invention makes it possible to significantly reduce the frictional forces on dried hair compared to the comparative composition, even though it comprises a lower silicone content.

A cationic surfactants/silicone ratio greater than or equal to 7 can therefore make it possible to significantly increase the efficiency of deposit of silicone on the hair and to obtain an added value regarding the sensory and disentangling qualities, and also a significant gain regarding the performance in terms of the criterion of smoothness of the hair.

### EXAMPLE 4

Composition G according to the invention and comparative composition G′ were prepared from the following ingredients (% AM):

TABLE 10

| | G | G′ |
|---|---|---|
| Amodimethicone (DOWSIL AP-8568 AMINO FLUID | 0.17 | 0.17 |
| from Dow Corning) | | |
| Behentrimonium chloride | 0.95 | 0.95 |
| Dipalmitoylethylhydroxyethylmonium methosulfate | 1.0 | 1.0 |
| Cocobetaine | 0.02 | — |
| Cetearyl alcohol | 4.6 | 4.6 |
| Cetyl esters | 0.4 | 0.4 |
| pH Agent (pH = 4.1 ± 0.5) | qs | qs |
| Preservatives | qs | qs |
| Water | qs 100 | qs 100 |

Compositions which can be used as a rinse-out hair care product (conditioner) are obtained.

### Deposit on Hair

The compositions are tested on locks of natural Caucasian hair of 2.7 g and 27 cm in length.

Firstly, the locks are cleaned using a standard silicone-free shampoo (DOP Camomile), in a proportion of 0.4 g/g of hair The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). Finally, the locks are dried for 30 minutes in an oven at 60° C.

The deposit of the silicone on the hair is quantified by X-ray fluorescence spectrometry via the measurement of the mass concentration of silicon element on the hair fiber. The amount of silicon naturally present in the untreated hair is subtracted from the measurement obtained on the treated hair (silicon deposit=content measured on treated hair−content measured on untreated hair).

The following results are obtained (silicon deposit measured by XRF on dry hair treated with compositions G or G′). The values are expressed in μg of silicon element per g of hair.

TABLE 11

| | G (invention) | G′ (comparative) |
|---|---|---|
| Silicon deposit | 220 | 194 |
| Standard deviation | 16 | 61 |

It is noted that the composition according to the invention results in a greater silicone deposit on the hair than the comparative composition.

### Sensory Evaluation

The tests are carried out on locks of medium-sensitized Caucasian hair (SA20) of 2.7 g and 27 cm in length. The locks are pre-cleaned using a standard shampoo (DOP Camomile) in a proportion of 0.4 g/g of hair.

Each block is then treated with a conventional repair shampoo (ELSEVE Total Repair 5, L'Oreal Paris) in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). The hair is wrung dry and then evaluated while wet. Finally, the locks are dried with a hairdryer and then evaluated (dry).

The disentangling is evaluated using a comb, whereas the smooth nature and the coating are scored on feel. The scores cover a scale of 0 (lack of performance) to 5 (maximum performance).

The following results are obtained:

TABLE 12

| | Evaluated criterion | G (invention) | G′ (comparative) |
|---|---|---|---|
| Wet hair | Ease of disentangling | 4.5 | 3.5 |
| | Smooth feel | 4 | 3 |
| | Coating | 4 | 3 |
| Dry hair | Smooth feel | 4 | 3 |
| | Coating | 4 | 3 |

It is noted that the composition according to the invention comprising the amphoteric surfactant is significantly superior, over the whole of the criteria tested, to the composition without amphoteric surfactant. This superiority manifests itself on wet hair through a greater ease of disentangling, hair that feels smoother and they coating that is more present signalling a better transformation of the fiber. On dried hair, the hair appears more treated (greater care coating) and softer (smoother feel).

### EXAMPLE 5

Composition H according to the invention and comparative composition H′ were prepared from the following ingredients (% AM):

TABLE 13

| | H | H′ |
|---|---|---|
| Amodimethicone (DOWSIL AP-8568 AMINO FLUID | 0.4 | 0.4 |
| from Dow Corning) | | |
| Behentrimonium chloride | 1.54 | 1.16 |
| Dipalmitoylethylhydroxyethylmonium methosulfate | 1.66 | 1.24 |
| Cocobetaine | 0.25 | 0.25 |
| Cetearyl alcohol | 4 | 4 |
| Cetyl esters | 0.4 | 0.4 |
| pH Agent (pH = 4.1 ± 0.5) | qs | qs |
| Preservatives | qs | qs |
| Water | qs 100 | qs 100 |
| Cationic surfactants/silicone ratio | 8 | 6 |

Compositions which can be used as a rinse-out hair care product (conditioner) are obtained.

### Sensory Evaluation

The tests are carried out on locks of medium-sensitized Caucasian hair (SA20) of 2.7 g and 27 cm in length. The locks are pre-cleaned using a standard shampoo (DOP Camomile) in a proportion of 0.4 g/g of hair.

Each block is then treated with a conventional repair shampoo (ELSEVE Total Repair 5, L'Oreal Paris) in a proportion of 0.4 g/g of hair. The shampoo is massaged on the lock in order to make it form a foam, before being left in for 15 seconds, then a step of rinsing with water is carried out for 10 seconds (flow rate: 300 l/h, temperature: 35° C.).

Secondly, the composition to be tested is applied to the wet hair in a proportion of 0.4 g/g of hair, before a leave-in time of 2 minutes followed by rinsing with water for 15 seconds (flow rate: 300 l/h, temperature: 35° C.). The hair is wrung dry and then evaluated while wet. Finally, the locks are dried with a hairdryer and then evaluated (dry).

The disentangling is evaluated using a comb (1 evaluator), whereas the smooth nature and the coating are scored on feel (3 evaluators). The scores cover a scale of 0 (lack of performance) to 5 (maximum performance).

The following results are obtained:

TABLE 14

| | Evaluated criterion | H (invention) | H′ (comparative) |
|---|---|---|---|
| Wet hair | Ease of disentangling | 5.0 | 4.0 |
| | Smooth feel | 4.4 ± 0.3 | 3.0 ± 0.0 |
| | Coating | 3.9 ± 0.3 | 3.0 ± 0.0 |
| Dry hair | Smooth feel | 4.0 ± 0.4 | 3.0 ± 0.0 |
| | Coating | 4.3 ± 0.3 | 3.0 ± 0.5 |

It is noted that the composition according to the invention is significantly superior, over the whole of the criteria tested, to the comparative composition.

It provides greater use qualities (ease of disentangling) and better cosmetic performance on wet hair and dried hair (smooth feel, coating).

1. A cosmetic composition comprising:
one or more surfactants chosen from anionic surfactants and amphoteric surfactants,
at least two cationic surfactants different from one another, and
one or more silicones,
the weight ratio between the total content of cationic surfactants and the total content of silicone(s) being greater than or equal to 7.

2. The composition as claimed in claim 1, wherein the cationic surfactants are chosen from the following compounds which are quaternary ammonium salts:
the compounds corresponding to the general formula (II) below: 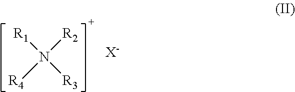
wherein:
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.
the groups R₁ to R₄, which may be identical or different, represent a linear or branched aliphatic group including from 1 to 30 carbon atoms, or an aromatic group such as aryl or alkylaryl, at least one of the groups R₁ to R₄ denoting a linear or branched aliphatic group including from 8 to 30 carbon atoms and preferably from 12 to 24 carbon atoms, the aliphatic groups possibly including heteroatoms notably such as oxygen, nitrogen, sulfur and halogens.
quaternary ammonium salts of imidazoline, such as those of formula (III): 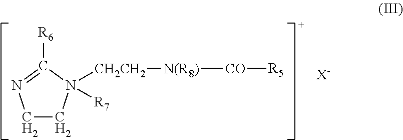
wherein R₅ represents an alkenyl or alkyl group comprising from 8 to 30 carbon atoms, for example derived from tallow fatty acids, R₆ represents a hydrogen atom, a C₁-C₄ alkyl group or an alkenyl or alkyl group comprising from 8 to 30 carbon atoms, R₇ represents a C₁-C₄ alkyl group, R₈ represents a hydrogen atom or a C₁-C₄ alkyl group and X⁻ is an anion chosen from the group of the halides, phosphates, acetates, lactates, alkyl sulfates, alkylsulfonates or alkylarylsulfonates, the alkyl and aryl groups of which preferably comprise, respectively, from 1 to 20 carbon atoms and from 6 to 30 carbon atoms.
quaternary diammonium or triammonium salts, in particular of formula (IV): 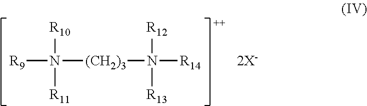
wherein
R₉ denotes an alkyl radical including approximately from 16 to 30 carbon atoms, which is optionally hydroxylated and/or optionally interrupted with one or more oxygen atoms,
R₁₀ is chosen from hydrogen or an alkyl radical including from 1 to 4 carbon atoms or a group (R₉ₐ)(R₁₀ₐ)(R₁₁ₐ)N—(CH2)3, with R₉ₐ, R₁₀ₐ, R₁₁ₐ, R₁₁, R₁₂, R₁₃ and R₁₄, which may be identical or different, chosen from hydrogen or an alkyl radical including from 1 to 4 carbon atoms, and
X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.
quaternary ammonium salts containing at least one ester function, such as those of formula (V) below: 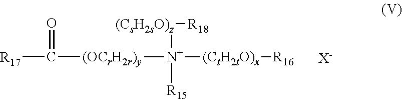
wherein:
R₁₅ is chosen from C₁-C₆ alkyl groups and C₁-C₆ hydroxyalkyl or dihydroxyalkyl groups;
R₁₆ is chosen from the group R19-C(O)—; groups R₂₀ which are linear or branched, saturated or unsaturated C₁-C₂₂ hydrocarbon-based groups; a hydrogen atom;
R₁₈ is chosen from the group R21-C(O)—; groups R₂₂ which are linear or branched, saturated or unsaturated C₁-C₆ hydrocarbon-based groups; a hydrogen atom;
R₁₇, R₁₉ and R₂₁, which may be identical or different, are chosen from linear or branched, saturated or unsaturated C₇-C₂₁ hydrocarbon-based groups;
r, s and t, which may be identical or different, are integers having values from 2 to 6;
y is an integer ranging from 1 to 10;
x and z, which may be identical or different, are integers having a value from 0 to 10;
X⁻ is a simple or complex, organic or mineral anion;
with the proviso that the sum x+y+z is from 1 to 15, that when x is 0 then R₁₆ denotes R₂₀, and that when z is 0 then R₁₈ denotes R₂₂.
fatty amines, and notably fatty amidoamines, preferably of formula (VI) below:
RCONHR″N(R′)2
wherein:
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C₅-C₂₉ and preferably C₇-C₂₃ alkyl radical, or a linear or branched C₅-C₂₉ and preferably C₇-C₂₃ alkenyl radical;
R″ represents a divalent hydrocarbon-based radical containing less than 6 carbon atoms, preferably from 2 to 4 carbon atoms and better still 3 carbon atoms; and
R′, which may be identical or different, represent a linear or branched, saturated or unsaturated and substituted or unsubstituted monovalent hydrocarbon-based radical containing less than 6 carbon atoms, preferably from 1 to 4 carbon atoms, preferably a methyl radical.

3. (canceled)

4. The composition as claimed in claim 1, comprising at least two cationic surfactants both chosen from those of formula (II), those of formula (V), those of formula (VI), and mixtures thereof, said formulae being those as defined in claim 2; better at least one of the cationic surfactants being chosen from those of formula (V) above, and at least one other of the cationic surfactants being chosen from those of formula (II) and/or (VI) above.

5. (canceled)

6. (canceled)

7. The composition as claimed in claim 1, comprising the cationic surfactants in a total amount ranging from 0.2% to 10% by weight, better still from 0.5% to 8% by weight, even better still from 1% to 5% by weight, relative to the total weight of the composition.

8. The composition as claimed in claim 1, comprising one or more additional anionic surfactants, preferably chosen, alone or as a mixture, from:
C₆-C₂₄ and in particular C₁₂-C₂₀ alkyl sulfates;
C₆-C₂₄ and in particular C₁₂-C₂₀ alkyl ether sulfates; preferably comprising from 1 to 20 ethylene oxide units;
C₆-C₂₄ and notably C₁₂-C₂₀ alkylsulfosuccinates;
C₆-C₂₄ and in particular C₁₂-C₂₀ olefin sulfonates;
C₆-C₂₄ and in particular C₁₂-C₂₀ alkyl ether sulfosuccinates;
(C₆-C₂₄)acylisethionates and preferably (C₁₂-C₁₈)acylisethionates;
C₆-C₂₄ and notably C₁₂-C₂₀ acylsarcosinates;
(C₆-C₂₄)alkyl ether carboxylates, preferably (C₁₂-C₂₀)alkyl ether carboxylates; in particular those including from 2 to 50 ethylene oxide groups;
polyoxyalkylenated (C₆-C₂₄)alkylamido ether carboxylic acids and salts thereof, in particular those including from 2 to 50 alkylene oxide and in particular ethylene oxide groups;
C₆-C₂₄ and in particular C₁₂-C₂₀ acylglutamates;
C₆-C₂₄ and in particular C₁₂-C₂₀ acylglycinates;
in particular in acid form or in the form of alkali metal or alkaline-earth metal, ammonium or amino alcohol salts;
preferentially chosen from C₆-C₂₄ and notably C₁₂-C₂₀ alkyl sulfates, C₆-C₂₄ and notably C₁₂-C₂₀ alkyl ether sulfates preferably comprising from 1 to 20 ethylene oxide units; these surfactants being more particularly in the form of alkali metal or alkaline-earth metal, ammonium or amino alcohol salts; and also mixtures thereof.

9. The composition as claimed in claim 1, comprising one or more anionic surfactants in a total amount of less than or equal to 1% by weight, better still (strictly) less than 1% by weight, notably being able to range from 0.001% to 1% by weight, notably from 0.005% to 0.5% by weight, better still from 0.01% to 0.3% by weight, relative to the total weight of the composition.

10. The composition as claimed in claim 1, comprising one or more amphoteric surfactants notably chosen, alone or as a mixture, from (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines;
optionally quaternized secondary or tertiary aliphatic amine derivatives having the respective structures (A), (B) and (C) below: 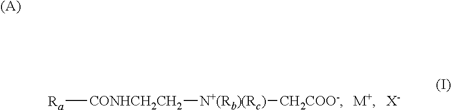
wherein:
Rₐ represents a C₁₀ to C₃₀ alkyl or alkenyl group derived from an acid RₐCOOH preferably present in hydrolyzed coconut kernel oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent; 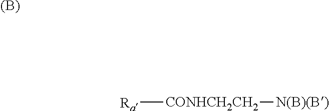
wherein:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid R_{a′}—COOH which is preferably present in coconut kernel oil or in hydrolyzed linseed oil, or an alkyl group, in particular a C₁₇ group, and its iso form, or an unsaturated C₁₇ group. 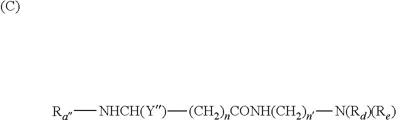
wherein:
Y″ represents the group —COOH, —COOZ″ or —CH₂—CH(OH)SO₃H or the group CH₂CH(OH)SO₃—Z″;
R_{d} and Rₑ, independently of one another, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in coconut kernel oil or in hydrolyzed linseed oil;
n and n′ denote, independently of one another, an integer ranging from 1 to 3.
preferably (C₈-C₂₀)alkylbetaines such as cocoylbetaine, (C₈-C₂₀)alkylamido(C₃-C₈)alkylbetaines such as cocamidopropylbetaine, and mixtures thereof, and the compounds of formula (C) such as the salts, notably the sodium salt of diethylaminopropyl laurylaminosuccinamate.

11. The composition as claimed in claim 1, wherein the amphoteric surfactant(s) are present in a total amount of less than or equal to 1% by weight, better still (strictly) less than 1% by weight, notably being able to range from 0.001% to 1% by weight, notably from 0.005% to 0.5% by weight, better still from 0.01% to 0.3% by weight, relative to the total weight of the composition.

12. The composition as claimed in claim 1, comprising a total content of surfactant(s), chosen from anionic and amphoteric surfactants, ranging from 0.001% to 1% by weight, notably from 0.005% to 0.5% by weight, better still from 0.01% to 0.3% by weight, relative to the total weight of the composition.

13. The composition as claimed in claim 1, comprising one or more silicones chosen from amino silicones, non-amino silicones, and mixtures thereof; notably chosen from nonvolatile liquid non-amino silicones, nonvolatile liquid amino silicones, and mixtures thereof.

14. The composition as claimed in claim 1, comprising one or more silicones chosen, alone or as mixtures, from the following amino silicones:
A) the polysiloxanes corresponding to formula (I): 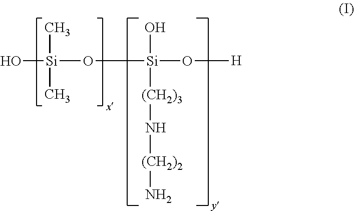
wherein x′ and y′ are integers such that the weight-average molecular mass (Mw) is between 5000 and 500 000 g/mol;
B) the amino silicones corresponding to formula (II): 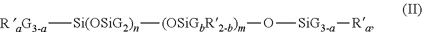
wherein:
G, which is identical or different, denotes a hydrogen atom or a phenyl, OH, C₁-C₈ alkyl, for example methyl, or C₁-C₈ alkoxy, for example methoxy, group;
a and a′, which may be identical or different, denote 0 or an integer from 1 to 3, in particular 0, with the proviso that at least one from among a and a′ is equal to zero,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
R′, which may be identical or different, denotes a monovalent radical of formula —C_{q}H_{2q}L wherein q is a number ranging from 2 to 8 and L is an optionally quaternized amino group chosen from the following groups: —NR″-Q-N(R″)₂, —N(R″)₂, —N⁺(R″)₃ A⁻, —N+H(R″)₂ A⁻, —N⁺H₂(R″) A⁻, —NR″-Q-N⁺(R″)H₂ A⁻, —NR″-Q-N⁺(R″)₂H A⁻ and —NR″-Q-N⁺(R″)₃ A⁻, wherein R″, which may be identical or different, denotes hydrogen, phenyl, benzyl or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, notably a halide, such as fluoride, chloride, bromide or iodide;
the amino silicones of formula (II) preferably being chosen from:
(i) the “trimethylsilyl amodimethicone” silicones corresponding to formula (III): 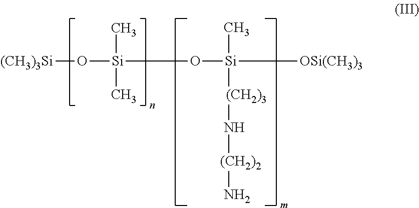
wherein m and n are numbers such that the sum (n+m) varies from 1 to 2000, preferably from 20 to 1000, notably from 50 to 600, better still from 50 to 150; it being possible for n to denote a number from 0 to 1999 and notably from 49 to 149 and it being possible for m to denote a number from 1 to 2000 and notably from 1 to 10;
(ii) the silicones of formula (IV) below: 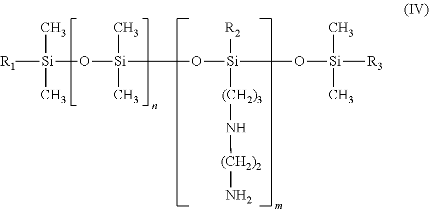
wherein:
m and n are numbers such that the sum (n+m) ranges from 1 to 1000, notably from 50 to 250 and more particularly from 100 to 200; n denoting a number from 0 to 999 and notably from 49 to 249 and more particularly from 125 to 175, and m denoting a number from 1 to 1000, notably from 1 to 10 and more particularly from 1 to 5; and
R₁, R₂ and R₃, which may be identical or different, represent a hydroxyl or C₁-C₄ alkoxy radical, at least one of the radicals R₁ to R₃ denoting an alkoxy radical;
(iii) the silicones of formula (V) below: 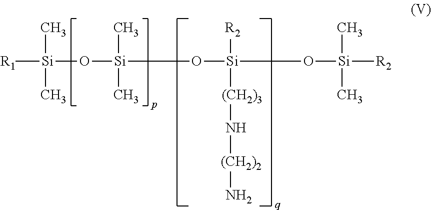
wherein:
p and q are numbers such that the sum (p+q) ranges from 1 to 1000, in particular from 50 to 350 and more particularly from 150 to 250; p denoting a number from 0 to 999, notably from 49 to 349 and more particularly from 159 to 239, and q denoting a number from 1 to 1000, notably from 1 to 10 and more particularly from 1 to 5; and
R₁ and R₂, which are different, represent a hydroxyl or C₁-C₄ alkoxy radical, at least one of the radicals R₁ or R₂ denoting an alkoxy radical;
(iv) the silicones of formula (VI) below: 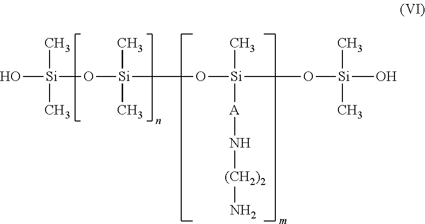
wherein:
m and n are numbers such that the sum (n+m) ranges from 1 to 2000 and notably from 50 to 150, n denoting a number from 0 to 1999 and notably from 49 to 149, and m denoting a number from 1 to 2000 and notably from 1 to 10; and
A denotes a linear or branched alkylene radical containing from 4 to 8 carbon atoms and preferably 4 carbon atoms; this radical is preferably linear;
(v) the silicones of formula (VII) below: 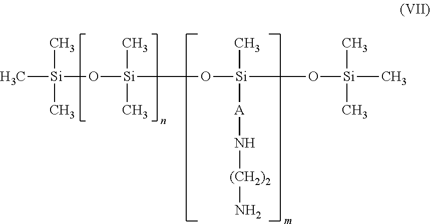
wherein:
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
A denotes a linear or branched alkylene radical containing from 4 to 8 carbon atoms and preferably 4 carbon atoms; this radical is preferably branched.
C) the amino silicones corresponding to formula (VIII): 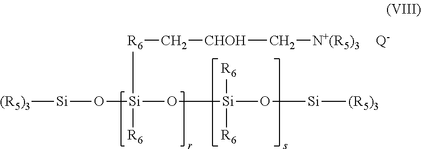
wherein:
R₅ represents a monovalent hydrocarbon-based radical having from 1 to 18 carbon atoms, and in particular a C₁-C₁₈ alkyl or C₂-C₁₈ alkenyl radical, for example methyl;
R₆ represents a divalent hydrocarbon-based radical, particularly a C₁-C₁₈ alkylene radical or a divalent C₁-C₁₈, for example C₁-C₈, alkyleneoxy radical linked to the Si via an SiC bond;
Q⁻ is an anion such as a halide ion, notably chloride, or an organic acid salt, notably acetate;
r represents a mean statistical value ranging from 2 to 20 and in particular from 2 to 8; and
s represents a mean statistical value ranging from 20 to 200 and in particular from 20 to 50.
D) the quaternary ammonium silicones of formula (IX): 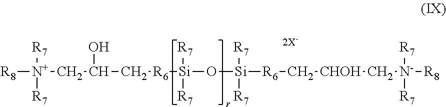
wherein:
R₇, which may be identical or different, represent a monovalent hydrocarbon-based radical having from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon-based radical, particularly a C₁-C₁₈ alkylene radical or a divalent C₁-C₁₈, for example C₁-C₈, alkyleneoxy radical linked to the Si via an SiC bond;
R₈, which may be identical or different, represent a hydrogen atom, a monovalent hydrocarbon-based radical having from 1 to 18 carbon atoms, and in particular a C₁-C₁₈ alkyl radical, a C₂-C₁₈ alkenyl radical or a —R₆—NHCOR₇ radical;
X⁻ is an anion such as a halide ion, notably chloride, or an organic acid salt, notably acetate; and
r represents a mean statistical value ranging from 2 to 200 and in particular from 5 to 100.
E) the amino silicones of formula (X): 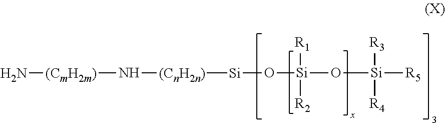
wherein:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;
F) the multiblock polyoxyalkylenated amino silicones, of (AB)ₙ type, A being a polysiloxane block and B being a polyoxyalkylenated block including at least one amine group; said silicones preferably being constituted of repeating units having the general formula below: 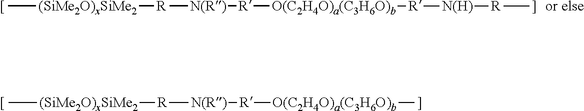
wherein:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C₂-C₁₂ hydrocarbon-based radical, optionally including one or more heteroatoms such as oxygen; preferably, R, which may be identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which may be identical or different, represent a linear or branched divalent C₂-C₁₂ hydrocarbon-based radical, optionally comprising one or more heteroatoms such as oxygen; preferably, R′, which may be identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.
G) the amino silicones of formulae (XI) and (XII): 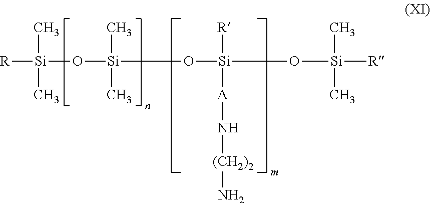
wherein:
R, R′ and R″, which may be identical or different, denote a C₁-C₄ alkyl group or a hydroxyl group,
A denotes a C₃ alkylene radical; and
m and n are numbers such that the weight-average molecular mass of the compound is between 5000 and 500 000; 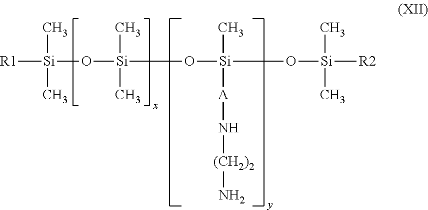
wherein:
x and y are numbers ranging from 1 to 5000; preferably, x ranges from 10 to 2000 and more preferentially from 100 to 1000; preferably, y ranges from 1 to 100;
R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.
h) polysiloxanes and notably polydimethylsiloxanes, including primary amine groups at only one chain end or on side chains, such as those of formula (XIV), (XV) or (XVI): 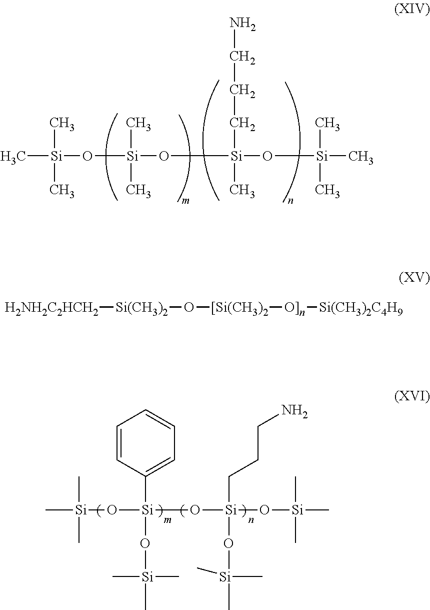
In formula (XIV), the values of n and m are such that the weight-average molecular mass of the amino silicone is between 1000 and 55 000.
In formula (XV), the value of n is such that the weight-average molecular mass of the amino silicone is between 500 and 3000.
In formula (XVI), the values of n and m are such that the weight-average molecular mass of the amino silicone is between 500 and 50 000.
I) the amino silicones corresponding to formula (XVIII) below: 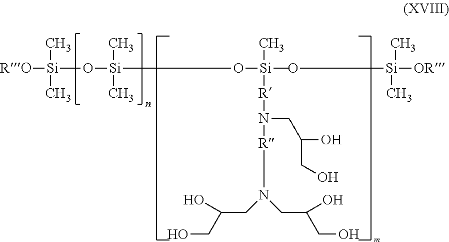
wherein:
n is a number between 1 and 1000, preferably between 10 and 500, better still between 25 and 100, even better still between 50 and 80;
m is a number between 1 and 200, preferably between 1 and 100, better still between 1 and 10 and even better still between 1 and 5;
R′″, which may be identical or different, preferably identical, are linear or branched, saturated or unsaturated alkyl radicals comprising from 8 to 30 carbon atoms, preferably 10 to 24 carbon atoms, notably 12 to 18 carbon atoms; it being possible for said radicals to optionally be substituted with one or more hydroxyl groups OH;
R′ is a linear or branched, divalent alkylene radical having from 1 to 6 carbon atoms, notably from 2 to 5 carbon atoms;
R″ is a linear or branched, divalent alkylene radical having from 1 to 6 carbon atoms, notably from 1 to 5 carbon atoms.
the amino silicone(s) preferably being chosen, alone or as a mixture, from the amino silicones of formulae (I), (III), (VI), (VII), (XI) and/or (XII) defined above, even better still from the amino silicones of formulae (I), (III) and/or (XI) as defined above.

15. The composition as claimed in claim 1, comprising the silicone(s) in a total content preferably ranging from 0.01% to 5% by weight, preferentially from 0.05% to 3% by weight, and more preferentially from 0.1% to 2.5% by weight, or even from 0.1% to 1% by weight, even better still from 0.1% to 0.8% by weight, relative to the total weight of the composition.

16. The composition as claimed in claim 1, wherein the weight ratio between the total content of cationic surfactants and the total content of silicone(s) present in the composition according to the invention ranges from 7 to 20, notably from 8 to 15 and even better still from 9 to 13.

17. The composition as claimed in claim 1, comprising one or more nonsilicone solid fatty substances, advantageously chosen from solid fatty alcohols, solid esters of fatty acids and/or of fatty alcohols, waxes, butters, ceramides, and mixtures thereof; better still chosen from solid fatty alcohols, solid esters of fatty acids and/or of fatty alcohols, and mixtures thereof; even better still from linear or branched, saturated, preferably linear and saturated, (mono)alcohols comprising from 8 to 40 carbon atoms, better still from 10 to 30, or even from 12 to 24 atoms, even better still from 14 to 22 carbon atoms; C₉-C₂₆ alkyl palmitates, C₉-C₂₆ alkyl myristates, C₉-C₂₆ alkyl stearates; and mixtures thereof.

18. A process for the cosmetic treatment of keratin materials, notably human keratin fibers such as the hair, comprising the application to said keratin materials of a composition as claimed in claim 1.

19. The composition as claimed in claim 1, comprising at least one cationic surfactant chosen from those of formula (II), those of formula (V), those of formula (VI), and mixtures thereof, said formulae being as defined in claim 2; better still from those of formula (II) or (V) above, and mixtures thereof; even better still from those of formula (V) above.

20. The composition as claimed in claim 1, comprising one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms;
better still, the composition comprising:
(i) one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms; and
(ii) one or more cationic surfactants chosen from:
(iii) tetraalkylammonium salts, notably dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, dicetyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts; preferably the chloride, bromide or methyl sulfate salts of these compounds; and/or
(iib) the amidoamines of formula (VI) RCONHR″N(R′)2 as defined above.

21. The composition as claimed in claim 1, comprising:
one or more cationic surfactants chosen from the salts, such as the chlorides, bromides or methosulfates, of diacyloxyethyldimethylammonium, of diacyloxyethylhydroxyethylmethylammonium, of monoacyloxyethyldihydroxyethylmethylammonium, of triacyloxyethylmethylammonium, of monoacyloxyethylhydroxyethyldimethylammonium and mixtures thereof; preferably of diacyloxyethyldimethylammonium and/or of diacyloxyethylhydroxyethylmethylammonium; the acyl groups preferably having 14 to 18 carbon atoms; and
one or more cationic surfactants chosen from tetraalkylammonium salts, notably dialkyldimethylammonium or alkyltrimethylammonium salts wherein the alkyl group includes from about 12 to 22 carbon atoms, in particular behenyltrimethylammonium, stearyltrimethylammonium, distearyldimethylammonium, dicetyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts; preferably the chloride, bromide or methyl sulfate salts of these compounds.
better still, the composition comprising:
one or more cationic surfactants chosen from the salts, notably the chlorides, bromides or methosulfates, of dipalmitoylethylhydroxyethylmethylammonium, and mixtures thereof;
and one or more cationic surfactants chosen from the salts, notably the chloride, bromide or methosulfates, of behenyltrimethylammonium, of distearyldimethylammonium, of cetyltrimethylammonium, of dicetyldimethylammonium and mixtures thereof.
